(19) United States
(12) Patent Application Publication
(10) Pub. No.: US 2024/0336954 A1
(43) Pub. Date: Oct. 10, 2024

(54) AUTOMATED DESIGN OF PRIMER SETS FOR NUCLEIC ACID AMPLIFICATION

(71) Applicant: Detect, Inc., Guilford, CT (US)

(72) Inventors: Jakob T. Nielsen, Tilst (DE); Mary O'Neill, Seattle, WA (US); Simon Chardin, Paris (FR); Henry Kemble, Paris (FR); Spencer Glantz, West Hartford, CT (US); Ben Goldstein, Westport, CT (US); Matt Higgins, London (GB); Morten Schaumburg, Aarhus V. (DE); Eric Kauderer-Abrams, Redwood City, CA (US)

(21) Appl. No.: 18/627,991

(22) Filed: Apr. 5, 2024

Related U.S. Application Data
(60) Provisional application No. 63/457,478, filed on Apr. 6, 2023.

(51) Int. Cl. C12Q 1/6811; C12N 15/10; G16B 25/20

(52) U.S. Cl. CPC C12Q 1/6811; C12N 15/1089; G16B 25/20

(57) ABSTRACT

Methods are provided for determining a primer set for amplifying a target nucleic acid, as well as apparatuses and computer-readable storage media configured to perform aspects of the methods. In some cases, the methods include obtaining a first primer set; generating multiple child primer sets by performing modifications to the first primer set; and, for each of the child primer sets, determining a fitness score of the child primer set and, if the fitness score is at or above a predetermined threshold, determining the child primer set to be an acceptable primer set and adding the child primer set to a collection of acceptable primer sets stored in a memory device. The generating may generate at least some of the child primer sets in parallel. Multiple collections of acceptable primer sets may be generated in parallel. Various aspects of the methods may be controlled by a genetic algorithm.

### CROSS-REFERENCE TO RELATED APPLICATIONS

The present application claims the benefit of priority of U.S. Provisional Application No. 63/457,478 filed Apr. 6, 2023, entitled “AUTOMATED DESIGN OF PRIMER SETS FOR NUCLEIC ACID AMPLIFICATION,” the entire contents of which is incorporated by reference herein.

### FIELD

The technology of the present invention relates generally to determination of sets of primers useable for nucleic acid amplification. More specifically, the present invention relates to methods and apparatuses for rapidly determining one set or multiple sets of primers useable to perform nucleic acid amplification tests (NAATs) for detecting the presence of a target nucleic acid.

### BACKGROUND

The ability to rapidly diagnose diseases-particularly highly communicable infectious diseases—is critical to preserving human health through early detection and containment of the infectious diseases. Rapid testing is critical to identifying infected individuals quickly and minimizing their interactions with others, in order to minimize the spread of the diseases. As one example, the high level of contagiousness, the high mortality rate, and the lack of an early treatment for the coronavirus disease 2019 (COVID-19) caused by Severe Acute Respiratory Syndrome Coronavirus 2 (SARS-CoV-2) have resulted in a pandemic that has already killed millions of people. The existence of rapid, accurate diagnostic tests, useable for detecting COVID-19 as well as other diseases, could allow individuals infected with a disease to be quickly identified and isolated, which could assist with containment of the disease. However, viral pathogens, such as SARS-CoV-2, are prone to continuous mutations resulting in the emergence of fast-spreading variants. In the absence of such diagnostic tests, diseases such as COVID-19 and its variants, which may result from natural mutations, may spread unchecked throughout communities.

### SUMMARY

Provided herein are apparatuses and methods for rapidly determining one or multiple primer sets useable to detect the presence of a target nucleic acid. The apparatuses and methods provided herein may be used to generate a collection of primer sets that may be optimized according to a genetic algorithm that takes into account oligonucleotide characteristics of the primers of each of the primer sets and that also takes into account second-order effects of interactions of various groups of two or more primers within each of the primer sets. The collection of primer sets may be filtered to reduce or eliminate the occurrence of false-positive detections of non-variant nucleic acids that are in the same family as the target nucleic acid, and also may be filtered to reduce or eliminate the occurrence of false-positive detections of nucleic acids corresponding to background substances that may be present in a sample containing the target nucleic acid.

According to a first aspect of the present technology, a method for determining a set of primers for amplifying a target nucleic acid is provided. The method may be comprised of: (a) obtaining a first primer set comprised of a plurality of primers; (b) generating a plurality of child primer sets by performing a plurality of modifications to the first primer set; (c) for each of the child primer sets, determining a fitness score of the child primer set; and (d) for each of the child primer sets, if the fitness score of the child primer set is at or above a predetermined threshold, determining the child primer set to be an acceptable primer set and adding the child primer set to a first collection of acceptable primer sets stored in a memory device.

In some embodiments of this aspect, the generating may generate at least some of the child primer sets in parallel.

In some embodiments of this aspect, the generating may generate at least some of the child primer sets one by one.

In some embodiments of this aspect, the generating may be performed until a total number of acceptable primer sets in the first collection is at or above a predetermined threshold.

In some embodiments of this aspect, the acceptable primer sets of the first collection may be stored in the memory device together with corresponding fitness scores of the acceptable primer sets.

In some embodiments of this aspect, the method may further be comprised of, if the fitness score of the child primer set is below the predetermined threshold, storing the child primer set and the fitness score of the child primer set in the memory device in a second collection of unacceptable primer sets.

In some embodiments of this aspect, the method may further be comprised of outputting the first collection for use in amplifying the target nucleic acid or for use in optimization of one or more acceptable primer sets of the first collection.

In some embodiments of this aspect, at least one of the child primer sets may be generated by changing a nucleotide position of a starting point or an ending point of one or more primers of the first primer set.

In some embodiments of this aspect, at least one of the child primer sets may be generated by causing a mutation in one or more primers of the first primer set.

In some embodiments of this aspect, at least one of the child primer sets may be generated by replacing one or more primers of the first primer set with one or more primers of a collection of candidate primers.

In some embodiments of this aspect, at least one of the child primer sets may be generated by combining one or more primers of another child primer set with one or more primers of the first primer set.

In some embodiments of this aspect, the method may further be comprised of: clustering the acceptable primer sets of the first collection into two or more groups of acceptable primer sets, each group of acceptable primer sets being comprised of primer sets having a common characteristic that is different from a characteristic of another group of acceptable primer sets; and, for each group of acceptable primer sets, culling the primer sets of the group so that no more than four primer sets remain in the group.

In some embodiments of this aspect, the obtaining of the first primer set may be comprised of: modifying an acceptable primer set of the first collection, or modifying a child primer set having a fitness score below the predetermined threshold.

In some embodiments of this aspect, the obtaining of the first primer set and/or the generating of the child primer sets may be controlled by a genetic algorithm.

In some embodiments of this aspect, (a) through (d) may be performed by at least one computer processor.

In some embodiments of this aspect, (a) through (d) may be performed a plurality of times by a plurality of computer processors, with at least one computer processor performing (a) through (c) concurrently with at least one other computer processor.

In some embodiments of this aspect, the method may further be comprised of repeating (a) through (d) a plurality of times to generate a plurality of first collections.

In some embodiments of this aspect, the obtaining of the first primer set may be comprised of selecting an acceptable primer set from the collection.

In some embodiments of this aspect, the obtaining of the first primer set may be comprised of selecting primers from a collection of candidate primers based on a target function of a genetic algorithm. In some embodiments, the selecting of the primers may be comprised of: selecting a first primer randomly, and, for each other primer other than the first primer, selecting the other primer based on an optimization of the target function using the first primer and each already-selected other primer. In some embodiments, a fitness score of a primer set being evaluated may be determined by applying a plurality of parameters corresponding to the primer set being evaluated to a multi-variable scoring function that simultaneously takes into consideration any two or more properties derived from oligo sequences of the primer set being evaluated, the scoring function being a part of the genetic algorithm. In some embodiments, a total number of variables in the scoring function may be in a range of 30 to 100, or in a range of 40 to 90, or in a range of 50 to 80, or in a range of 60 to 70. In some embodiments, the two or more properties may include two or more of: an average linguistic sequence complexity of one or more primers of the primer set being evaluated, a melting point (Tₘ) of one or more primers of the primer set being evaluated, a difference in melting points (ΔTₘ) between a highest-Tₘ primer and a lowest-Tₘ primer of the primer set being evaluated, a percentage of guanine (G) and cytosine (C) nucleotides in one or more primers of the primer set being evaluated, a first Gibbs free energy (ΔG₁) for polymerase initiation of one or more primers of the primer set being evaluated, a difference between ΔG₁ and a Gibbs free energy (ΔGₓ) for polymerase initiation of one or more nucleic acids different from the target nucleic acid, a hybridization probability for one or more primers of the primer set being evaluated, and a genomic positional separation between two neighboring primers of the primer set being evaluated.

In some embodiments of this aspect, the method may further be comprised of determining the collection of candidate primers based on: a target genome sequence of the target nucleic acid, and a plurality of variant genome sequences of a plurality of variant nucleic acids, each of the variant nucleic acids being a variant of the target nucleic acid. In some embodiments, the determining of the collection of candidate primers may be based on a plurality of non-variant genome sequences of a plurality of non-variant nucleic acids, the non-variant genome sequences being comprised of: sequences belonging to a same family as the target nucleic acid and being a non-variant of the target nucleic acid, and sequences belonging to families of common organisms unrelated to the target nucleic acid. In some embodiments, the determining of the collection of candidate primers may be comprised of: determining, based on the variant genome sequences, a plurality of first conserved regions of the target genome sequence, and determining single primers corresponding to the first conserved regions, determining, based on the non-variant genome sequences, a plurality of second conserved regions of the target genome sequence, and determining single primers corresponding to the second conserved regions, and determining a collection of single primers that are single primers for the first conserved regions and that are not single primers for the second conserved regions, the collection of single primers being the collection of candidate primers.

In some embodiments of this aspect, the method may further be comprised of preparing a pre-screening pipeline for the target nucleic acid by performing at least one of: collecting assemblies of genome sequence data comprised of a plurality of genome sequences associated with the target nucleic acid; performing pan genome analysis on at least some of the genome sequences of the genome sequence data to determine at least one measure of diversity, identifying plasmids in the assemblies of genome sequence data; selecting one or more of the genome sequences to be representative of the target nucleic acid, and preparing a summary file of information summarizing the one or more of the genome sequences selected to be representative of the target nucleic acid; and identifying homologs of the one or more of the genome sequences selected to be representative of the target nucleic acid. In some embodiments, the at least one measure of diversity may be comprised of any one or any combination of: a Watterson's Theta value corresponding to a number of segregating sites, a Pi value corresponding to a value for pairwise nucleotide diversity, and a Tajima's D value corresponding to a neutrality test statistic. In some embodiments, the pre-screening pipeline may be prepared prior to execution of (a). In some embodiments, the target nucleic acid may be a bacterial genomic target. For example, the bacterial genomic target may be Chlamydia trachomatis or Neisseria gonorrhoeae.

According to another aspect of the present technology, an apparatus for determining a set of primers for amplifying a target nucleic acid is provided. The apparatus may be comprised of: a computer system comprised of at least one processor; and a memory device coupled to the computer system. The computer system may be programmed to: (a) obtain a first primer set comprised of a plurality of primers, (b) generate a plurality of child primer sets by performing a plurality of modifications to the first primer set, (c) for each of the child primer sets, determine a fitness score of the child primer set, and (d) for each of the child primer sets, if the fitness score of the child primer set is at or above a predetermined threshold, determine the child primer set to be an acceptable primer and add the child primer set to a first collection of acceptable primer sets stored in the memory device.

In some embodiments of this aspect, the computer system may be comprised of a plurality of processors. Each of the processors may be configured to perform (a) through (d) separately such that the computer system may generate a plurality of first collections.

In some embodiments of this aspect, the computer system may be programmed to implement one or more features of the foregoing method of the first aspect.

According to another aspect of the present technology, a non-transitory computer-readable storage medium is provided in which is stored code that, when executed by one or more processors of a computer system, implements a method for determining a set of primers for amplifying a target nucleic acid. The method may be comprised of: (a) obtaining a first primer set comprised of a plurality of primers; (b) generating a plurality of child primer sets by performing a plurality of modifications to the first primer set; (c) for each of the child primer sets, determining a fitness score of the child primer set; and (d) for each of the child primer sets, if the fitness score of the child primer set is at or above a predetermined threshold, determining the child primer set to be an acceptable primer set and adding the child primer set to a first collection of acceptable primer sets stored in a memory device.

In some embodiments of this aspect, the generating may generate at least some of the child primer sets concurrently.

In some embodiments of this aspect, the generating may generate at least some of the child primer sets one by one.

In some embodiments of this aspect, the generating may be performed until a total number of acceptable primer sets in the first collection is at or above a predetermined threshold.

In some embodiments of this aspect, the method may further be comprised of storing in the memory device the acceptable primer sets of the first collection together with corresponding fitness scores of the acceptable primer sets.

In some embodiments of this aspect, the method may further be comprised of, if the fitness score of the child primer set is below the predetermined threshold, adding the child primer set and the fitness score of the child primer to a second collection of unacceptable primer sets stored in the memory device.

In some embodiments of this aspect, the method may further be comprised of: clustering the acceptable primer sets of the first collection into two or more groups of acceptable primer sets, each group of acceptable primer sets being comprised of primer sets having a common characteristic that is different from a characteristic of another group of acceptable primer sets; and, for each group of acceptable primer sets, culling the primer sets of the group so that no more than four primer sets remain in the group.

In some embodiments of this aspect, (a) through (d) may be performed a plurality of times by the one or more processors to generate a plurality of first collections.

In some embodiments of this aspect, the method may further be comprised of one or more features of the foregoing method of the first aspect.

### BRIEF DESCRIPTION OF THE DRAWINGS

A skilled artisan will understand that the accompanying drawings are for illustration purposes only. It is to be understood that in some instances various aspects of the present technology may be shown exaggerated or enlarged to facilitate an understanding of the invention. In the drawings, like reference characters generally refer to like features, which may be functionally similar and/or structurally similar elements, throughout the various figures. The drawings are not necessarily to scale, as emphasis is instead placed on illustrating and teaching principles of the various aspects of the present technology. The drawings are not intended to limit the scope of the present teachings in any way.

FIG. 1 shows a flow chart of actions performed to determine a set of primers for detecting a target nucleic acid sequence, according to some embodiments of the present invention.

FIG. 2A shows a block diagram of a computer system that may be configured with hardware and/or software to determine sets of primers for detecting a target nucleic acid sequence, according to some embodiments of the present technology.

FIG. 2B shows a flow chart for a method of performing an automated design technique, according to some embodiments of the present technology.

FIG. 2C shows a flow chart for a primer-set determination procedure, according to some embodiments of the present technology.

FIG. 2D shows a flow chart for an acceptable-primer-set determination procedure, according to some embodiments of the present technology.

FIG. 3A shows a flow diagram summarizing procedures of a genetic algorithm, according to some embodiments of the present technology.

FIG. 3B shows a flow diagram of a design method for improvement of primer design through experimental evaluation and machine learning, according to some embodiments of the present technology.

FIG. 4 shows a chart illustrating observed values vs. predicted values for inverse time-to-positivity values for “InitGen” or a collection of initial-generation primer sets.

FIG. 5 shows charts illustrating an average time to positivity (Tₚ) for different collections of primer sets and samples containing different amounts of a target pathogen to be detected by the collections of primer sets.

FIG. 6 shows a chart illustrating a comparison of successful amplifications for NextGen, InitGen, and Alternative primer sets.

FIG. 7 shows a chart illustrating exclusivity fraction as a function of same-family maximum genome identity for InitGen, NextGen, and Alternative primer sets.

FIGS. 8A-8D show diagrams of locations of LAMP primer sets on particular genes, in particular, POP7 (FIG. 8A), PPIA (FIG. 8B), ACTB (FIG. 8C), and GAPDH (FIG. 8D).

FIGS. 9A-9E show pre-screening pipeline results for Chlamydia trachomatis (CT). FIG. 9A shows the presence and absence of 1081 genes among 171 assemblies. Note that a tree was created using a binary presence and absence of accessory genes and therefore may not be reliable other than to roughly group isolates together based on their accessory genomes. FIG. 9B shows a pie-chart distribution of genes found in 100% of isolates (core), 99% of isolates (99th), 95% of isolates (soft), or less than 95% of isolates (accessory). Among genes with ≥150 bp with 90% of the coding sequence containing non-missing data, distributions of (FIG. 9C) Watterson's theta, (FIG. 9D) nucleotide diversity (Pi), and (FIG. 9E) Tajima's D are plotted.

FIGS. 10A-10E show pre-screening pipeline results for Neisseria gonorrhoeae (NG). FIG. 10A shows the presence and absence of 4173 genes among 864 assemblies. Note that a tree was created using a binary presence and absence of accessory genes and therefore may not be reliable other than to roughly group isolates together based on their accessory genomes. FIG. 10B shows a pie-chart distribution of genes found in 100% of isolates (core), 99% of isolates (99th), 95% of isolates (soft), or less than 95% of isolates (accessory). Among genes with ≥150 bp with 90% of the coding sequence containing non-missing data, distributions of (FIG. 10C) Watterson's theta, (FIG. 10D) nucleotide diversity (Pi), and (FIG. 10E) Tajima's D are plotted.

FIGS. 11A-11E show pre-screening pipeline results for Streptococcus pyogenes (GAS). FIG. 11A shows the presence and absence of 7276 genes among 2185 assemblies. Note that a tree was created using a binary presence and absence of accessory genes and therefore may not be reliable other than to roughly group isolates together based on their accessory genomes. FIG. 11B shows a pie-chart distribution of genes found in 100% of isolates (core), 99% of isolates (99th), 95% of isolates (soft), or less than 95% of isolates (accessory). Among genes with ≥150 bp with 90% of the coding sequence containing non-missing data, distributions of (FIG. 11C) Watterson's theta, (FIG. 11D) nucleotide diversity (Pi), and (FIG. 11E) Tajima's D are plotted.

### DETAILED DESCRIPTION

### 1. Introduction

Nucleic acid amplification tests (NAATs) are sensitive tests that may be used to diagnose a pathogen by detecting small amounts of genetic material corresponding to the pathogen. NAATs typically depend on a set of primers, or nucleic acid segments matching a portion of the genome of the genetic material, to detect the genetic material. The genetic material's genome may be formed of a large number of nucleic-acid bases, any portion of which may be used to form a primer. For example, the SARS-CoV-2 virus may have a genome comprised of over 29,000 bases, and primers for this virus may have various different sizes and may be formed from various different regions or segments of the genome, with some primers being formed from genome segments that may overlap with genome segments corresponding to other primers, and with some primers being formed from genome segments that may be far apart from genome segments corresponding to other primers. Therefore, the number of different sets or combinations of two or more primers may be huge, especially for genomes having tens of thousands of bases or more. As will be appreciated, searching the numerous combinations to find an optimal set of primers, which is able to detect the genetic material with high specificity, high sensitivity, and high speed, among various desirable traits, could entail significant resources to evaluate the numerous combinations.

Diagnostic tests face the challenge of targeting the most conserved regions of the genome of a pathogen (e.g., a virus, bacterium, fungus, parasite, etc.) of interest to be detected, without cross-reacting with a genome of a different pathogen, which may lead to an erroneous or false positive detection of the pathogen of interest. For example, for the SARS-CoV-2 virus, diagnostic tests typically target regions on the virus' genome that are both conserved or common within the SARS-CoV-2 virus and its variants and, at the same time, are distinct from other viruses (e.g., other types of coronaviruses such as Severe Acute Respiratory Syndrome (SARS), Middle East Respiratory Syndrome (MERS), etc.).

Existing approaches to finding an optimal set of primers for a NAAT typically use a set of guidelines that is determined anecdotally. For example, an initial primer may be selected based on how its properties (e.g., length, melting temperature, etc.) compare with those established in the set of guidelines. One by one, k additional primers may then be selected based on the initial primer and other previously selected primers, if any. Once k primers have been selected for the set, the set may be evaluated for its performance in detecting a target genome. The initial primer may be the basis for a number of different sets of primers, with the set showing the best results being chosen as the optimal primer set for the NAAT. The inventors have recognized and appreciated that such an optimization approach may be flawed, because the approach assumes that the primer selected to be the initial primer is to be among the primers of the best primer set for the NAAT. The inventors have recognized and appreciated that this approach does not take into account second-order effects where, for example, the synergistic effects of a combination of primers that do not include the initial primer may lead to better results than the best results in which the initial primer is included.

The inventors have developed techniques, described herein, that efficiently determine an optimized set of primers from a collection of potential primers for a target genome, and that efficiently determine a collection of optimized sets of primers for detecting the target genome. In some embodiments of the present technology, various procedures of the techniques may be performed sequentially by one or more computer processors, as described below. In some embodiments of the present technology, various procedures of the techniques may be performed in parallel by one or more processors, as described below.

The present disclosure provides methods and apparatuses for determining sets of primers useable to detect a particular nucleic acid, such as the nucleic acid corresponding to a particular pathogen (e.g., a particular virus, or bacteria, or fungus, or parasite, or other). The nucleic acid of interest, to be detected, may be referred to herein as the “target” nucleic acid. Each primer of a set of primers may be used to amplify a particular sequence of nucleotides of the target nucleic acid. The primer sets determined by the techniques described herein may be used to perform NAATs for life-sciences research (e.g., to investigate therapies for treating an ailment being studied) and also for use in products aimed at identifying whether a subject is afflicted with an ailment associated with a particular known pathogen. NAATs may be used to identify a particular pathogen of interest, such as a particular virus, by detecting traces of specific genetic material of the pathogen, such as nucleic acid sequences (ribonucleic acid (RNA) and/or deoxyribonucleic acid (DNA)), through chain reactions of enzymatic amplification.

Polymerase chain reaction (PCR) technology is used in some NAATs. PCR-based diagnostic tests generally have been preferred over traditional antibody-based diagnostic tests, sometimes referred to as antigen tests, because of the relatively high sensitivity and high accuracy of PCR tests, which may amplify a very small amount of a nucleic acid sequence of a pathogen and therefore may detect the presence of the pathogen at an early stage of the pathogen-borne disease (e.g., when a very small amount of the pathogen is present in a sample taken from a subject being evaluated for the disease). NAATs can use different methods to amplify nucleic acids, including but not limited to reverse-transcription PCR (RT-PCR) methods and isothermal amplification-based methods. Isothermal amplification-based methods may include, but are not limited to, nicking endonuclease amplification reaction (NEAR) methods, transcription mediated amplification (TMA) methods, loop-mediated isothermal amplification (LAMP) methods, recombinase polymerase amplification (RPA) methods, nucleic acid sequence-based amplification (NASBA) methods, rolling circle amplification (RCA) methods, exponential amplification reaction (EXPAR) methods, helicase-dependent amplification (HDA) methods, clustered regularly interspaced short palindromic repeats (CRISPR) methods, and strand displacement amplification (SDA) methods.

Conventional NAATs, such as PCR-based tests, typically are performed in laboratory settings due to the need for expensive laboratory equipment to be used, whereas isothermal NAATs may be performed in laboratory settings or in point-of-care settings (e.g., medical clinics, doctors' offices, etc.) or in non-clinical settings (e.g., homes, schools, etc.) because expensive laboratory equipment need not be used. For example, RT-PCR technology typically requires temperature cycling and elevated-temperature steps that are performed in laboratories by trained technicians. As such, diagnostic tests using conventional NAATs with RT-PCR technology may be costly and may be associated with a time delay of several hours to several days before results may be obtained. In contrast, isothermal NAATs may be more robust alternatives to conventional NAATs and have become increasingly popular because they generally do not require expensive heating equipment to perform, e.g., thermal cycling, and consequently may be less costly than conventional NAATs. Moreover, isothermal NAATs may be used in non-laboratory settings and, without the need for thermal cycling, may be relatively rapid in comparison with diagnostic testing using RT-PCR-based conventional NAATs. For example, a rapid diagnostic test for detection of the SARS-CoV-2 virus using the isothermal amplification technique RT-LAMP has been developed by Detect, Inc. (Guilford, Connecticut, US). This test, known as Detect Covid-19 Test™, may be administered (or self-administered) by a lay person in a non-laboratory setting, with results being obtainable in less than two hours.

According to some embodiments of the present technology disclosed herein, a design system is provided for generating primer sets for NAATs. In some embodiments, the design system may be comprised of methods that may be used to generate primer sets for NAAT reactions. In some embodiments, the methods may be performed by or facilitated by one or more computer processors. In some embodiments, the design system may be comprised of apparatuses that may be used to generate primer sets for NAATs. In some embodiments, the apparatuses may be comprised of one or more computer processors. As noted above, various techniques of the present technology may be performed in parallel; the one or more computer processors may be used advantageously to improve efficiency in this regard.

As noted above, the primer sets generated by the design system may be used to detect specific genetic material. In some embodiments of the present technology, the primer sets may be used to detect a nucleic acid of interest. For example, the primer sets may be used to detect a DNA sequence of interest and/or an RNA sequence of interest. In other embodiments, the DNA and/or RNA sequence of interest detected by the primer sets may correspond to a specific pathogen and therefore may be used to diagnose an ailment associated with that pathogen.

In some embodiments, the primer sets designed by the design system may be used in NAATs that may be performed in point-of-care (POC) settings, which may include non-laboratory settings as well as laboratory settings. Such NAATs may be used, in some embodiments, in diagnostic tests performable by lay persons who do not have training in laboratory procedures or techniques.

NAATs typically use a set of primers. Each primer may be an oligonucleotide sequence complementary to a specific segment of a nucleic acid sequence of a genome of interest. The binding of one or more primers to the genome of interest may initiate an action of an enzyme (e.g., a polymerase) that may be important for amplification of the genome of interest and/or may stabilize an intermediate structure that may be important for amplification of the genome of interest. As will be appreciated, a NAAT having a relatively larger number of primers and/or relatively longer primers may be relatively more accurate in detecting a target nucleic acid sequence with fewer errors (e.g., erroneous or “false-positive” results) than a NAAT having a relatively fewer number of primers and/or relatively shorter primers. However, as the number of primers increases and/or as the lengths of the primers increase, the relative complexity of testing may increase and the speed of obtaining results may decrease. A NAAT may be designed to utilize a particular number of primers and a particular combination of primers based on various considerations (e.g., efficient amplification, specificity, speed, etc.). For example, some considerations in designing primer sets may include: characteristics of conserved regions of the genome of the target nucleic acid and its variants, a percentage of known variants having the conserved regions, whether a suitable number of the conserved regions have a length in a desired length range, and whether the conserved regions include regions that are found in non-variant nucleic acids, which may lead to undesirable cross-reactivity with a non-variant nucleic acid or other background genetic material, leading to false positive detection results.

According to some embodiments of the present technology, the design system may be configured to generate a large number of candidate primer sets. In some embodiments, multiple primer sets (e.g., at least 10, at least 100, at least 500, at least 1000) may be generated in parallel. The primer sets generated by the design system may be specific to detecting a target genome, i.e., a target nucleic acid of interest. In some embodiments, the number of primer sets generated for detection of a target genome may be different from the number of primer sets generated for detection of another target genome. In some embodiments, the target genome may be comprised of genetic material of a virus, or a bacterium, or a fungus, or a parasite, or another type of pathogen. In some embodiments, the design system may generate primer sets that minimize or avoid cross-reactivity with a non-variant nucleic acid (e.g., a nucleic acid of a genome in the same family as the target genome but not a variant of the target genome). In some embodiments, the design system may generate primer sets that minimize or avoid cross-reactivity with background genetic material that may be present in biological samples. In some embodiments, the design system may generate primer sets corresponding to conserved regions common to the target genome and most, if not all, known variants of the target genome.

Some considerations when designing efficient primer sets may include various physico-chemical properties of the primers of the primer sets, including but not limited to nucleotide composition and thermodynamic parameters, discussed below. The inventors have recognized that current guidelines for designing primer sets may be inherently biased due to initial assumptions that permeate most if not all procedures, thus causing some guidelines to be too strict and some guidelines to be too loose. For example, conventional automated primer-design techniques typically are deterministic and based on assumptions regarding an initial primer, with such assumptions staying in effect for all primer sets generated from the initial primer. Such assumptions, however, may neglect second-order effects between pairs of primers that do not include the initial primer. Additional information on conventional guidelines for designing primer sets may be found in, e.g., Notomi T et al., Nucleic Acids Res. 2000 Jun. 15, 28 (12): 63-63; Nagamine K et al., Mol Cell Probes. 2002 Jun. 16 (3): 223-9 (4-5). Additional information on conventional automated primer design techniques for NAATs may be found in, e.g., Higgins M et al., Bioinformatics. 2019 Feb. 15, 35 (4): 682-4; Mitani Y et al., Nat Methods. 2007 Mar. 4 (3): 257-62 (6-7).

One amplification technique that has gained more and more interest is LAMP, which is an isothermal technique that may be used to amplify a target nucleic acid in less than two hours, and in some cases less than 60 minutes, with high sensitivity. Existing software tools for LAMP-related primer-set design include PrimerExplorer V5 (Eiken Chemical Co., Ltd., Japan); NEB® Primer Design Tools (New England BioLabs, Inc., Ipswich, MA, US); LAVA, which is an open-source approach to designing primer sets; LAMP Designer (PREMIER Biosoft International, San Francisco, CA, US); and GLAPD. The inventors have recognized that conventional techniques for generating primer sets do not adequately take into consideration two important factors in primer design: conservation of regions of the target nucleic acid and distinctiveness of regions of the target nucleic acid. These factors are discussed below. Instead, conventional techniques rely on manual selection of one or more highly conserved target regions, which can be difficult to find for highly diverse targets, such as in the case of an influenza virus, which may have a scarcity of conserved regions in common with its variants.

### 1.1 Overview of Primer-Set Design Technology

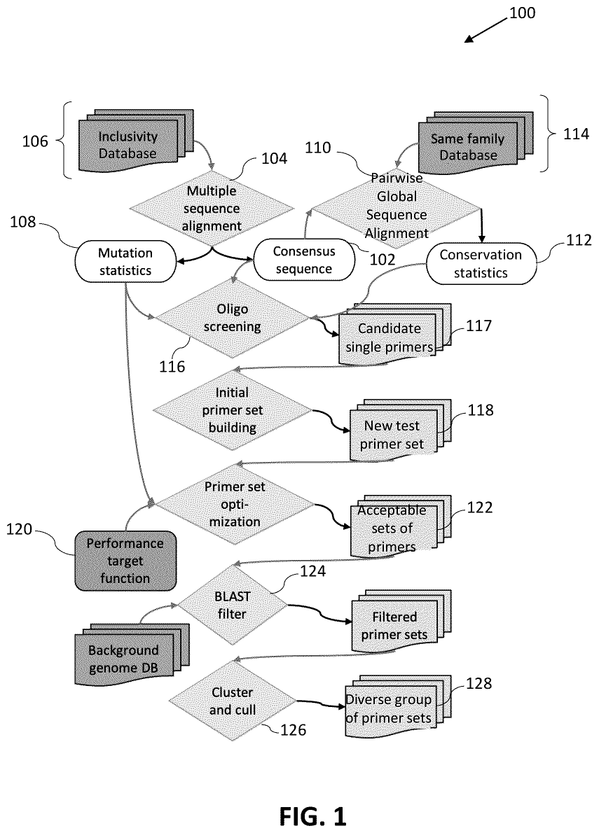

FIG. 1 schematically shows an overview diagram for a method 100 of designing a set of primers for amplifying a target nucleic acid (also referred to as “target genome” herein), according to some embodiments of the present technology. Various details of the method 100 are discussed below. In some embodiments, the method 100 may be comprised of obtaining a consensus sequence 102. The consensus sequence 102 may be a nucleic-acid sequence for a target genome to be amplified. In some embodiments, once a pathogen for a disease has been isolated, sequencing of the pathogen's genome may be performed a plurality of times to determine, from the plurality of sequencing results, a genomic sequence most likely to be the pathogen's genomic sequence. In some embodiments, the plurality of sequencing results may be stored in a database and analyzed to determine which genomic sequence is most likely to be the pathogen's genomic sequence. For example, the pathogen's genomic sequence may be determined to correspond to a sequence having the highest probability of being accurate, out of the plurality of sequencing results under consideration. In some embodiments, the consensus sequence 102 may correspond to the sequence having the highest probability of being accurate for the pathogen (i.e., the target genome). In some embodiments, the consensus sequence 102 may be obtained from a reputable laboratory (e.g., the Centers for Disease Control and Prevention (CDC) in Atlanta, Georgia, US). In some embodiments, the consensus sequence 102 may be determined by a private laboratory and agreed upon by the scientific community to represent the pathogen. In some embodiments, the consensus sequence 102 may be uploaded to a computer system and stored in a memory accessible by one or more processors of the computer system, to be used as a reference in one or more procedures of the method 100 to design primer sets for the target genome. In some embodiments, the consensus sequence 102 may be used to define all potential primers for the target genome, with the primers being continuous segments of oligonucleotides having a range of lengths between minimum and maximum thresholds.

In some embodiments, the consensus sequence 102 may be derived from a multiple sequence alignment (MSA) procedure 104. In some embodiments, the MSA procedure 104 may be comprised of performing one or more MSAs on data from an inclusivity database 106. In some embodiments, the data in the inclusivity database 106 may be comprised of the sequencing results discussed above and/or a plurality of sequences of variant genomes (also referred to as “variant sequences” herein). The data in the inclusivity database 106 may be input by a user and stored in a local memory accessible by the computer system or may be accessed by the computer system from an external data storage facility via a communication network (e.g., the Internet). In some embodiments, the MSA procedure 104 may be used to derive mutation statistics 108, which may include statistics describing, of a total number of variant sequences in the inclusivity database 106, what fraction of the variant sequences match the target genome for any given position on the sequence for the target genome (i.e., the consensus sequence).

In some embodiments, the consensus sequence 102 may be used in combination with information stored in a same family database 114. In some embodiments, a pairwise global sequence alignment procedure 110 may be performed on the information in the same family database 114 in conjunction with the consensus sequence 102 to derive conservation statistics 112. In some embodiments, the same family database 114 may contain a plurality of sequences of non-variant genomes in the same family as the target genome. In some embodiments, sequence information of the non-variant genomes may be useful to avoid false-positive detections of genomes that are related to the target genome but are not variants of the target genome. In some embodiments, the conservation statistics 112 may be statistics describing commonality or conservation relative to the consensus sequence. For example, the conservation statistics 112 may include statistics on conserved regions of the consensus sequence 102 in common with one or more variant sequences as well as a percentage of all the variant sequences sharing each conserved region, and/or may include statistics on conserved regions of the consensus sequence 102 in common with one or more non-variant sequences as well as a percentage of all the non-variant sequences sharing each conserved region, and/or may include statistics on conserved regions of the consensus sequence 102 in common with one or more variant sequences and one or more non-variant sequences. In some embodiments, the non-variant genome sequences may be input by a user and stored in the same family database 114 in a local memory accessible by the computer system or may be stored in an external data storage facility accessible by the computer system via a communication network (e.g., the Internet).

In some embodiments of the present technology, the consensus sequence 102, the mutation statistics 108, and the conservation statistics 112 may be used as data for various procedures of the method 100. In some embodiments, the consensus sequence 102, the mutation statistics 108, and the conservation statistics 112 may be used in an oligo screening procedure 116 to produce a plurality of candidate single primers 117, as discussed below.

In some embodiments of the present technology, a new test primer set 118 may be chosen from the plurality of candidate single primers 117. In some embodiments, a first primer of the new test primer set 118 may be selected randomly, and the subsequent primers may be added at suitable nearby positions on the consensus sequence of the target genome. For example, a separation distance of a predetermined number of nucleotides may be taken into consideration in selecting the subsequent primers. In some embodiments, the new test primer set 118 may be built by adding primers one-by-one. In some embodiments, a multi-variable performance target function 120 may be used as a predictive measure to optimize the new test primer set 118 to create an acceptable set of primers 122. In some embodiments, the performance target function 120 may be considered a performance prediction function. Variable parameters of the performance target function 120 may be comprised of oligo characteristics (e.g., length, melting temperature (Tₘ), G+C content, etc.) of the primers in the new test primer set 118, as discussed below. For example, optimization of the new test primer set 118 may be comprised of calculating a first fitness score for the new test primer set 118 based on the performance target function 120; varying a structural characteristic of the new test primer set 118 and calculating a second fitness score; varying another structural characteristic of the new test primer set 118, and calculating a third fitness score; etc. The new test primer set 118 may be varied a predetermined number of times to arrive at a predetermined number of fitness scores. A primer set corresponding to a best fitness score amongst all the fitness scores may be designated an acceptable set of primers 122 determined from the new test primer set 118. The procedure described above for the new test primer set 118 may be performed multiple times (e.g., 100, 1000, 5000, 10,000, etc.) for multiple new test primer sets, and multiple acceptable sets of primers determined from the multiple new test primer sets may be included with the acceptable set of primers 122 as part of a collection of acceptable sets of primers for the target genome. Fitness-score calculations are discussed in more detail below.

In some embodiments, the collection of acceptable sets of primers for the target genome may be filtered to remove one or more sets of primers that may have an undesirable cross-reactivity with background substances that may be present in samples bearing the pathogen to be detected. By filtering out sets of primers that may have homology with genomic sequences of background substances, the occurrence of false-positive detection results may be reduced. In some embodiments, filtering against homology with genomic sequences of background substances may be performed by a BLAST filter 124, to produce a filtered collection of acceptable sets of primers.

In some embodiments of the present technology, a diverse group of primer sets 128 may be produced by a clustering and culling procedure 126, which may be comprised of clustering or segregating the filtered collection of acceptable sets of primers into groups of like sets and, for each group of like sets, culling all but one representative primer set to represent the group. Thus, the diverse group of primer sets 128 may be comprised of primer sets that are diverse from each other. In some embodiments, the diverse group of primer sets 128 may be output as an optimized set of primers for amplifying the target genome.

In some embodiments, the diverse group of primer sets 128 may be an initial generation of primer sets whose efficacy in detecting a target genome is evaluated in order to fine tune or improve variables and functional expressions of the performance target function 120, which may be considered an initial-generation performance target function, to produce a next-generation performance target function. In some embodiments, in a next-generation evaluation, a new diverse group of primer sets may be generated using the next-generation performance target function. Efficacy of the new diverse group of primer sets, i.e., the next generation of primer sets, in detecting the target genome may be evaluated and compared with the efficacy of the initial generation of primer sets to determine whether modifications to the initial performance target function made noticeable improvements to detection characteristics such as, e.g., detection time, detection sensitivity, etc. As will be appreciated, another “next generation” evaluation may be performed to further improve the next-generation performance target function based on detection data acquired from evaluating the diverse group of primer sets generated using the next-generation performance target function.

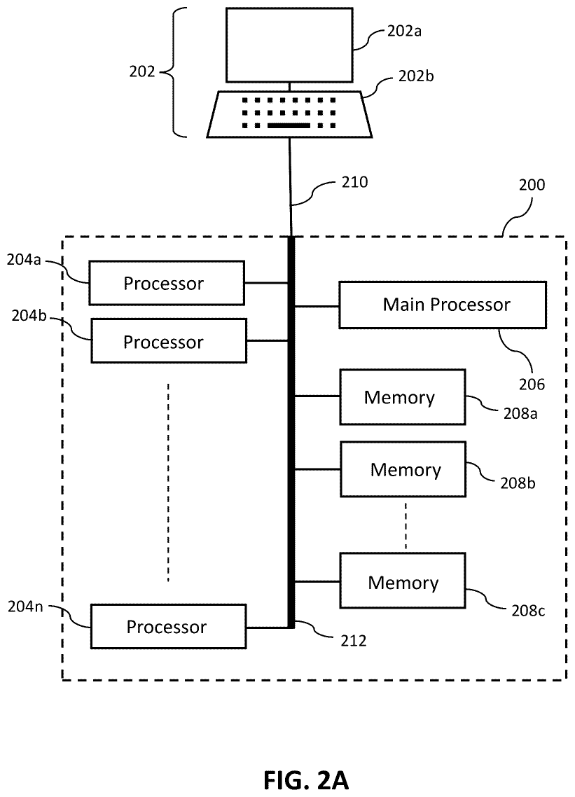

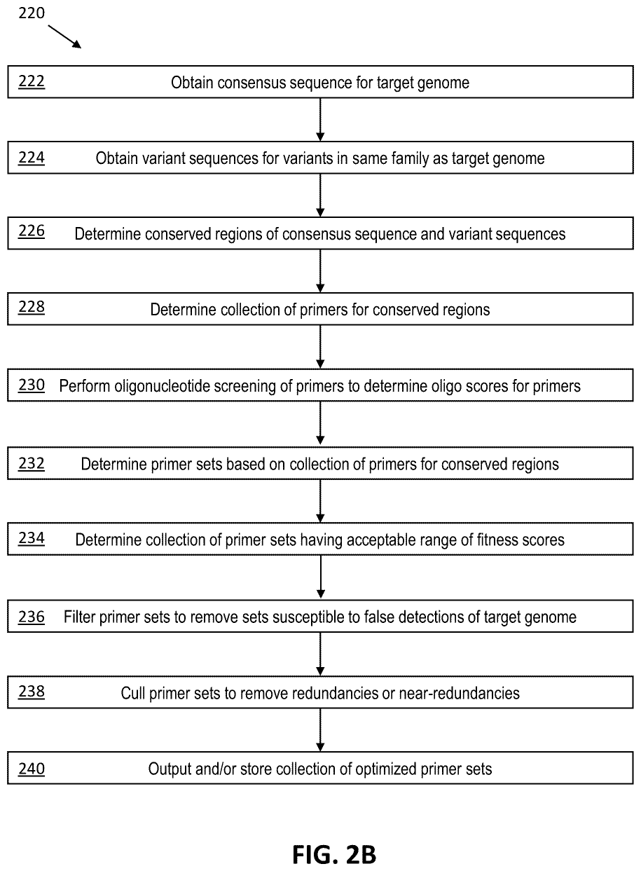

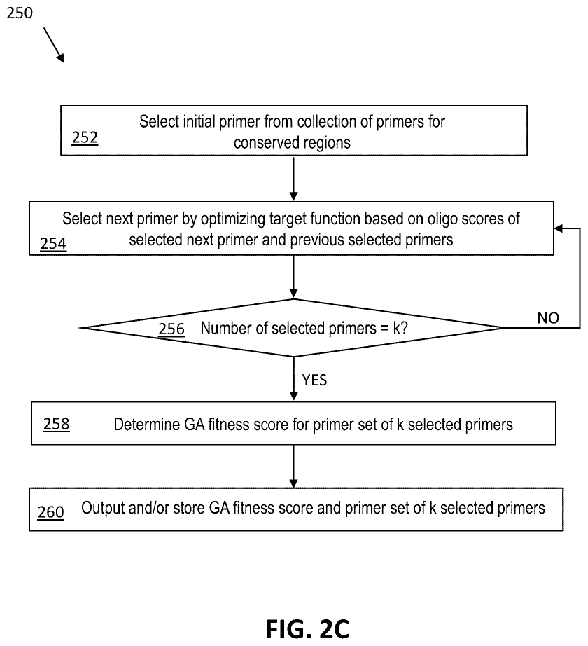

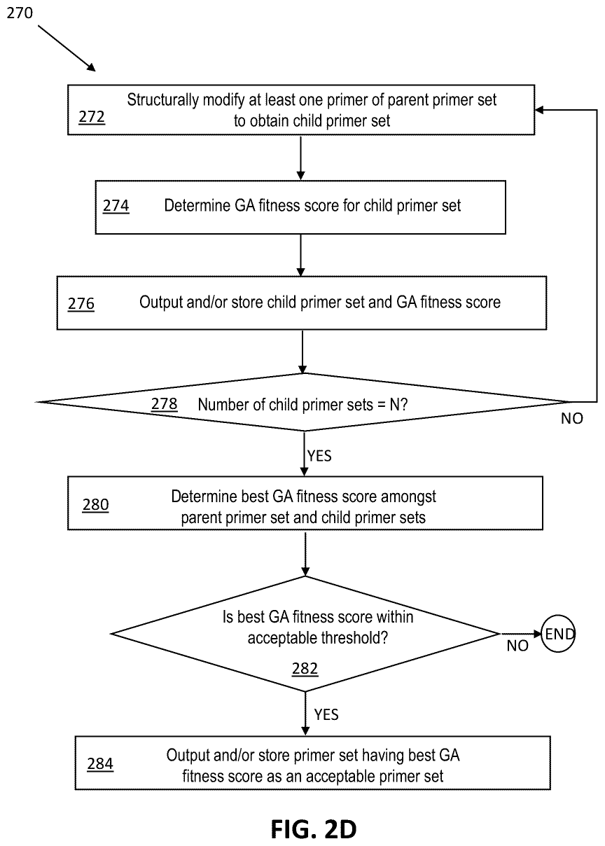

FIG. 2A schematically shows a design system 200, according to some embodiments of the present technology. In some embodiments, the design system 200 may be operably coupled, by wire or wirelessly, to an input terminal 202 configured to enable a user to input instructions and/or data to the design system 200 and/or to review queries and/or data generated by the design system 200. For example, the input terminal 202 may be comprised of a display 202a on which queries and/or data from the design system 200 may be viewed by the user, and also may be comprised of an input device 202b via which instructions and/or data may be provided to the design system 200. Examples of devices that may be used as the input device 202b include any one or any combination of: a keyboard, a pointing device (e.g., a mouse, a touch pad, a digitizing tablet, etc.); an input/output interface through which data and/or instructions stored on a non-transitory computer-readable storage medium may be downloaded and/or uploaded (e.g., a USB port, a hard-disk drive, etc.); and a microphone. In some embodiments, a communication interface may be provided through which data and/or instructions may be transferred via a communication network 210 (e.g., the Internet, a LAN, a WAN, etc.); etc. The communication interface may enable wired and/or wireless transmissions via known communication technologies. Examples of wireless protocols that may be used for communication include, but are not limited to: Wi-Fi (e.g., any of the IEEE 802.11 family of protocols), Bluetooth®, Zigbee and other IEEE 802.15.4-based protocols, cellular protocols, and the like. The microphone may be coupled to a speech-recognition module of the design system 200 to enable spoken data/instructions to be recognized and processed. In some embodiments, the design system 200 and the input terminal 202 may be operably interconnected, by wire or wirelessly, via the communication network 210, as shown in FIG. 2A.

In some embodiments of the present technology, the design system 200 may be comprised of one or more computer processors 204a, 204b, . . . 204n configured to control one or more aspects of an automated design technique for designing primer sets, as described herein. In some embodiments, the processors 204a, 204b, . . . 204n may be comprised of multiple central processing units (CPUs) that may be formed of multiple physically distinct integrated circuits (ICs) connected to each other, by wire or wirelessly, by an interconnection system 212. In some embodiments, the interconnection system 212 may be comprised of at least one communication bus and/or the communication network 210. In some embodiments, the interconnection system 212 may interconnect some or all electronic components of the design system 200. In some embodiments, the processors 204a, 204b, . . . 204n may be comprised of a multi-core processor that is a single IC configured with multiple cores or CPUs. In some embodiments, the processors 204a, 204b, . . . 204n may be comprised of a group of CPUs configured to operate in parallel to perform parallel processing of one or more procedures of the automated design technique, as discussed herein. In some embodiments, the processors 204a, 204b, . . . 204n may be comprised of a main processor 206 configured to control one or more CPUs of the processors 204a, 204b, . . . 204n, individually or in groups of two or more CPUs, to perform one or more procedures of the automated design technique.

In some embodiments, the design system 200 may be comprised of one or more memory devices 208a, 208b, . . . 208n operably coupled to the processors 204a, 204b, . . . 204n via the interconnection system 212. The memory devices 208a, 208b, . . . 208n may be comprised of any memory circuitry that is able to store data. For example, the memory devices 208a, 208b, . . . 208n may be comprised of any one or any combination of: a hard-drive memory (e.g., solid-state-memory drive, magnetic memory, optical-disk drive, etc.), a removable storage medium (e.g., flash/USB memory, optical disk, floppy disk, portable magnetic memory, etc.), and the like. The memory devices 208a, 208b, . . . 208n may be comprised of persistent and non-persistent memory devices formed of physical or tangible structures able to store computer-executable code in a non-transitory state.

In some embodiments of the present technology, techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that may be executed on a framework or virtual machine. Such computer-executable instructions may be stored on at least one non-transitory computer-readable storage medium, such as one or more those described above, and may be executed by the computer processors 204a, 204b, . . . 204n to perform various aspects of the techniques described herein.

In some embodiments, the memory devices 208a, 208b, . . . 208n may store code for executing various procedures of the automated design technique for designing primer sets and/or may store data used by various procedures of the automated design technique and/or may store data generated during execution of various procedures of the automated design technique, as discussed herein. For example, the main processor 206 may be configured to control the design system 200 according to executable code accessed from the memory devices 208a, 208b, . . . 208n. The executable code may be run or executed by one or more CPUs of the processors 204a, 204b, . . . 204n and the main processor 206 to control various electronic components and/or software routines of the design system 200. In some embodiments, the memory devices 208a, 208b, . . . 208n may be comprised of at least one non-transitory computer-readable storage medium storing modules of code for controlling a plurality of acts of the automated design technique, including acts shown in FIGS. 2B-2D.

FIG. 2B shows a flow chart for a method of performing an automated design technique 220, according to some embodiments of the present technology. It should be understood that the acts described herein for the automated design technique 220 need not be performed in the order shown, and some acts may be performed concurrently and/or in a different order than shown in FIG. 2B. In some embodiments, some or all acts of the automated design technique 220 may be performed entirely by a computer system (e.g., by the processors 204a, 204b, . . . 204n operating in conjunction with the memory devices 208a, 208b, . . . 208n).

At act 222, a consensus sequence for a target genome is obtained, as described herein. For example, the target genome may correspond to a nucleic-acid sequence for a pathogen to be detected via a NAAT. In some embodiments, the consensus sequence may have been derived previously and stored in the memory devices 208a, 208b, . . . 208n. In some embodiments, the consensus sequence may be derived from an analysis of a collection of potential nucleic-acid sequences to determine a most likely sequence for the target genome. The most likely sequence derived from the analysis may be stored in the memory devices 208a, 208b, . . . 208n as the consensus sequence representing the target genome, for further use in the automated design technique 220. In some embodiments, act 222 may be comprised of a computer processor (e.g., any of the processors 204a, 204b, . . . 204n) accessing the consensus sequence from a memory device (e.g., any of the memory devices 208a, 208b, . . . 208n).

At act 224, variant sequences, i.e., nucleic acid sequences of variants of the target genome, are obtained. The variant sequences may be variants of the target genome that are in the same family of pathogens as the target genome. In some embodiments, the variant sequences may be obtained and stored in the memory devices 208a, 208b, . . . 208n for further use in the automated design technique 220. In some embodiments, the variant sequences may be stored in an external database and act 224 may be comprised of uploading and storing the variant sequences. In some embodiments, act 224 may be comprised of a computer processor (e.g., any of the processors 204a, 204b, . . . 204n) accessing the variant sequences from a memory device (e.g., any of the memory devices 208a, 208b, . . . 208n). In some embodiments, although not shown in FIG. 2B, act 224 may include obtaining and storing non-variant sequences, i.e., nucleic-acid sequences of organisms that are in the same family as the target genome but are not variants of the target genome. In some embodiments, although not shown in FIG. 2B, act 224 may include obtaining and storing nucleic-acid sequences of background substances that may appear in samples that include the target genome. For example, if a sample is obtained from blood of a subject to be tested, the background substances may include substances commonly found in blood or in procedures for extracting blood from subjects.

At act 226, a computer processor (e.g., any of the processors 204a, 204b, . . . 204n) may analyze the consensus sequence and the variant sequences to determine conserved regions (“first conserved regions”), which may be sequence segments that are common to one or more of the variant sequences and the consensus sequence. In some embodiments, a computer processor (e.g., any of the processors 204a, 204b, . . . 204n) may analyze the consensus sequence and the non-variant sequences to determine conserved regions (“second conserved regions”), which may be sequence segments that are common to one or more of the non-variant sequences and the consensus sequence. At act 226, a set of conserved regions may be determined for the consensus sequence by filtering the first conserved regions to remove conserved regions corresponding to the second conserved regions, to minimize the possibility of a false-positive detection of a non-variant of the target genome.

At act 228, a collection of primers is obtained by determining a primer for each of the conserved regions of the set of conserved regions determined at act 226 or for each of a subset of the set of conserved regions determined at act 226.

At act 230, oligonucleotide (“oligo”) screening is performed on the collection of primers determined at act 228. In some embodiments, the oligo screening may be comprised of filtering the collection of primers to remove primers that are not within a predetermined range of lengths. For example, a primer may be removed if a number of nucleotides is less than a minimum number of nucleotides (e.g., 10 or 12 or 15 or 18 minimum nucleotides) or greater than a maximum number of nucleotides (e.g., 20 or 23 or 27 or 30 maximum nucleotides). As will be appreciated, priming stability may depend on competing factors. While relatively longer primers or primers with higher G+C content may be associated with an increased target affinity, such primers may also result in a slower reaction time. In contrast, relatively shorter primers or primers with lower G+C content may be associated with a decreased target affinity, such primers may result in a quicker reaction time. In some embodiments, the oligo screening may be comprised of associating each primer with any one or any combination of oligo characteristics for the primer: linguistic complexity; G+C content (e.g., a percentage of G and C nucleotides in the primer); thermodynamic properties (e.g., Gibbs free energy (ΔG), enthalpy (ΔH), entropy (ΔS); melting temperature (Tₘ); and hybridization probabilities for each base pair. The linguistic complexity may be defined as the number of distinct sub-nucleotide-sequences contained in the primer divided by the maximum theoretical number for this quantity. Primers with a linguistic complexity that is too low may be prone non-specific reactions both with target and non-target DNA. As will be appreciated, the oligo screening may be comprised of associating each primer with other characteristics not specifically listed herein.

In some embodiments, the characteristics associated with each primer may be used to determine kinetic properties for reactions involving the primer.

At act 232, primer sets are determined from the collection of primers for the conserved regions for the consensus sequence. In some embodiments, each primer set may be comprised of a predetermined number of primers (e.g., 4 primers, 5 primers, 6 primers, 7 primers, or 8 primers), with the primers of the primer set conforming with predetermined primer-set specifications (e.g., a specification of a separation distance between adjacent primers, a specification of melting point of one or more primers of the primer set, etc.).

FIG. 2C shows a flow chart for a primer-set determination procedure 250, according to some embodiments of the present technology. The procedure 250 may be used to determine one or more primer sets for act 230 of the automated design technique 220. At act 252, an initial primer is selected from a collection of primers for conserved regions. In some embodiments, the collection of conserved regions may be the set of conserved regions determined at act 226. In some embodiments, the initial primer may be selected randomly. In some embodiments, the initial primer may be selected based on one or more oligo characteristics. At act 254, a next primer of the primer set is selected based on oligo characteristics of the initial primer and any other primer previously selected for the primer set. In some embodiments, selection of the next primer may be comprised of optimizing a target function of a multi-variable genetic algorithm (GA) based on the oligo characteristics of the initial primer and the other primer(s) previously selected for the primer set. For example, oligo characteristics that may be taken into consideration may include any one or any combination of: first-order or linear differences in an oligo characteristic between the current constituents of the primer set being built (e.g., a difference in melting temperatures (ΔTₘ) between various pairs of primers); second-order or quadratic differences in an oligo characteristic between the current constituents of the primer set being built (e.g., a quadratic difference in melting temperatures (ΔTₘ²) between various pairs of primers); free energies of formation (e.g., AGs) for heterodimer formations; genomic positional separation between two primers; and LAMP-related free energies of formation for a forward inner primer (FIP) formation, a backward inner primer (BIP) formation, and homodimer formation. At act 256, after the next primer is selected, a decision is made as to whether the number of primers selected for the primer set has reached a desired number of primers, k. If the decision is NO, i.e., if it is decided that the number of primers has not yet reached k, the procedure 250 returns to act 254. If the decision is YES, i.e., if it is decided that the number of primers has reached k, the procedure 250 proceeds to act 258 where a fitness score is determined for the primer set of k primers. In some embodiments, oligo characteristics of the k primers of the primer set may be used as parameters of the GA to determine the fitness score for the primer set. At act 260, the primer set and its fitness score may be stored (e.g., in any of the memory devices 208a, 208b, . . . 208n) for later processing and/or the primer set may be output for further processing. In some embodiments, the GA may be comprised of the performance target function 120.

According to some embodiments of the present technology, the primer-set determination procedure 250 may be performed multiple times to determine a plurality of primer sets. These primer sets may be referred to herein as candidate primer sets. For example, the number of candidate primer sets determined by the procedure 250 may total 10 or 100 or 200 or 500 or 1000 or 5000, etc. When a relatively large number of primer sets is desired, the procedure 250 may be performed by a plurality of CPUs in parallel. On the other hand, when a relatively small number of primer sets is desired, the procedure 250 may be performed one at a time.

According to some embodiments of the present technology, each of the primer sets determined by the procedure 250 may serve as a “parent” primer set from which one or more “child” primer sets are generated. The parent and child primer sets may be used to determine an acceptable primer set that takes into consideration second-order effects of the primers in the parent primer set, as discussed below.

Returning to FIG. 2B, at act 234, the primer sets determined at act 232 may be analyzed to determine a collection of primer sets having an acceptable range of fitness scores. In some embodiments, the oligo characteristics of each primer may be used as input for the GA to determine the acceptability of each primer set.

FIG. 2D shows a flow chart for an acceptable-primer-set determination procedure 270, according to some embodiments of the present technology. At act 272, a child primer set is generated by structurally modifying at least one primer of a parent primer set, which may be a primer set determined by the procedure 250, in some embodiments. For example, a child primer set may be obtained by shifting a position of one or more primers of the parent primer set and/or by changing a length of one or more primers of the parent primer set, a child set may be a mutated version of one of the primers of the parent primer set, or a child set may be the result of crossover events between two parent primer sets, keeping some primers from the first parent primer set and other primers from the second parent primer set. At act 274, a fitness score is determined for the child primer set. For example, oligo characteristics of the primers of the child primer set may be used as parameters of the GA to determine the fitness score for the child primer set. At act 276, the child primer set and its fitness score may be stored (e.g., in any of the memory devices 208a, 208b, . . . 208n) for later processing and/or the child primer set may be output for further processing. At act 278, a decision is made as to whether the number of child primer sets generated from the parent primer sets has reached a desired number of child primer sets, N. If the decision is NO, i.e., if it is decided that the number of child primer sets has not yet reached N, the procedure 270 returns to act 272. If the decision is YES, i.e., if it is decided that the number of child primer sets has reached N, the procedure 270 proceeds to act 280 where all the child primer sets and the parent primer set are evaluated to determine which of the primer sets has the best fitness score (e.g., a score that minimizes a function of the GA). At act 282, a decision is made as to whether the best fitness score is within an acceptable range of fitness scores (e.g., the best fitness score meets a predetermined threshold for an acceptable score). If the decision is NO, i.e., if the best fitness score is not within the acceptable range, the procedure 270 ends and neither the parent primer set nor any of the child primer sets is considered acceptable for use in detecting the target genome. If the decision is YES, i.e., if the best fitness score is within the acceptable range, at act 284, the primer set corresponding to the best fitness score is determined to be an acceptable primer set and is output and/or stored (e.g., in any of the memory devices 208a, 208b, . . . 208n) for further processing.

According to some embodiments of the present technology, the acceptable-primer-set determination procedure 270 may be performed multiple times for multiple different parent primer sets to determine a plurality of acceptable primer sets. For example, the number of acceptable primer sets determined by the procedure 270 may total 10 or 100 or 200 or 500 or 1000 or 5000, etc. When a relatively large number of primer sets is desired, the procedure 270 may be performed by a plurality of CPUs in parallel. On the other hand, when a relatively small number of primer sets is desired, the procedure 270 may be performed one at a time. In some embodiments, the acceptable primer sets may be the collection of primer sets determined at act 234.

Returning to FIG. 2B, at act 236, the acceptable primer sets may be filtered to remove primer sets that may be susceptible to false detections of the target genome. In some embodiments, each acceptable primer set may be evaluated against non-variant sequences (e.g., nucleic acid sequences of genomes that are in the same family as the target genome but are not variants of the target genome), to determine a false-detection-likelihood value for detecting one or more of the non-variant sequences. In some embodiments, each acceptable primer set may be evaluated against sequences for known substances that may be background substances found in samples containing the target genome, to determine a false-detection-likelihood value for detecting one or more of the known background substances. If an acceptable primer set is determined to have a false-detection-likelihood value that exceeds a predetermined threshold, the acceptable primer set may be removed from further consideration.

At act 238, the acceptable primer sets may be compared with each other to cull some of the primer sets, to obtain a collection of optimized primer sets that are diverse from each other. In some embodiments, the accept primer sets may be segregated into a plurality of cluster groups, with each cluster group sharing at least one commonality. Examples of commonalities may include, but are not limited to, the positional distance. Each cluster group may be culled to remove all but one representative primer set for the cluster group.

At act 240, the representative primer sets for the different cluster groups may be output and/or stored as a collection of optimized primer sets for detecting the target genome. For example, the collection of optimized primer sets may be stored in any of the memory devices 208a, 208b, . . . , 208n.

### 2. Conserved Segments of Nucleic Acid of Target Genome and Determination of Initial Primers

### 2.1 Introduction

In some embodiments of the present technology, the design system generates a set of primers that may be efficient at amplifying and detecting a target genome as well as the most possible variant genomes of the target genome sequence, while possibly not cross-reacting to (i.e., falsely detecting) non-variant genomes or other genomes (e.g., background-substance genomes). In some embodiments, a set of candidate primers may be derived by the design system using at least three factors: a consensus sequence representing the target genome, mutation statistics relative to the consensus sequence, and conservation statistics relative to the consensus sequence. In some embodiments, the mutation statistics and the consensus sequence may be derived from multiple sequence alignments (MSA) performed on sequences in an inclusivity database, which may include sequences corresponding to a plurality of variant genomes. The sequences in the inclusivity database may be input by one or more users of the inclusivity database, which may be a private database or a publicly shared database. In some embodiments, the consensus sequence and sequences in a same family database, which may include sequences corresponding to non-variant genomes in the same family as the target genome, may be used to generate a global sequence alignment that produces the conservation statistics. In some embodiments, the conservation statistics, the mutation statistics, and the consensus sequence may be used for oligo screening to generate a plurality of candidate single primers, which may be primers that have been screened based on each primer's oligonucleotide characteristics. In some embodiments, a candidate single primer may be defined as a continuous oligonucleotide segment. In some embodiments, the oligonucleotide segment may have a complexity or length within a length range defined by a minimum threshold length and a maximum threshold length. In some embodiments, the length range may be between a minimum of 15 nucleotides and a maximum of 27 nucleotides, or between 10 and 35 nucleotides, or between 12 and 30 nucleotides, or between 8 and 40 nucleotides, or between 18 and 25 nucleotides, or between 20 and 45 nucleotides. In further embodiments, a candidate single primer may be defined by the 5′ and 3′ positions on the target genome (or on the consensus sequence used to represent the target genome, as discussed below). In some embodiments, a candidate single primer may contain segments from a sense strand of the target genome. In some embodiments, the candidate single primer may contain segments from an antisense strand of the target genome.

### 2.2 Consensus Sequence

As noted above, a consensus sequence may be used to represent a target genome to be amplified. The consensus sequence may be a genomic sequence most likely to accurately represent the target genome, out of a plurality of possible sequences under consideration for the target genome. In some embodiments, the consensus sequence may be obtained from the CDC or another a reputable laboratory. In some embodiments, the consensus sequence may be uploaded to a computer system and stored in a memory accessible by one or more processors of the computer system, to be used as a reference to design primer sets for the target genome. In some embodiments, the consensus sequence may be derived, as discussed below.

In some embodiments of the design system, the consensus sequence may be adapted from a target genome sequence (e.g., some or all of a known genome sequence for a pathogen) and may be used as a reference to derive a plurality of candidate single primers. In some embodiments, a primer may be a segment of nucleotides that matches a segment of the consensus sequence, and a candidate primer may be a segment of nucleotides that matches a segment of the consensus sequence and also matches a segment of each of at least one variant sequence for as many variant sequences as possible and at as many nucleotide positions as possible.

### 2.3 Variant Sequences

In some embodiments, the consensus sequence may be derived based on data in an inclusivity database, which may include a plurality of variant genome sequences. In some embodiments in which the target genome sequence is not known, the plurality of variant genome sequences may come from sequenced samples that may or may not include the genome sequence for a pathogen of interest (i.e., the target genome sequence). In such embodiments, the variant genome sequences may be aligned by MSA processing, and a result of the MSA processing may be used to generate the consensus sequence. In some other embodiments, the target genome sequence may be known, and a result of the MSA processing of the target genome sequence with the variant genome sequences may be used to modify the target genome sequence to remove insertions and deletions, to arrive at the consensus sequence.

In some embodiments, the MSA processing may be performed using commercially available or publicly available alignment tools. In some embodiments, an alignment tool may be chosen based on the diversity of the variant genome sequences relative to each other and/or relative to a known target genome sequence for which a consensus sequence is to be determined.

In some embodiments, in a case where there is less diversity amongst a known target genome sequence and its variant genome sequences, the target genome sequence may be aligned with the variant genome sequences using an alignment tool that maps each variant genome sequence pairwise to the target genome sequence. For example, an alignment tool that may be used for a target genome sequence having less diversity with its variants is NextAlign within Nextclade, which is publicly available and accessible at https://docs.nextstrain.org/projects/nextclade/en/stable/user/nextalign-cli.html. Results of such alignment may be used to determine a consensus sequence for the target genome sequence.

In some embodiments, in a case where there is more diversity amongst a known target genome sequence and its variant genome sequences, the target genome sequence may be aligned with its variants de novo with a MSA tool such as MAFFT, which is publicly available and accessible at https://mafft.cbrc.jp/alignment/software/.

In some embodiments, in a case wherein the target genome sequence is not known, the consensus sequence may be determined by aligning the variant genome sequences with each other, using an alignment tool chosen based on the relative diversity of the variant genome sequences.

### 2.4 Mutation and Conservation

In some embodiments, after determination of the consensus sequence, alignment results may be used to remove insertions (i.e., inserted sequence segments) from the variant genome sequences, to facilitate comparison of each variant genome sequence to the consensus sequence. In some embodiments, a variant genome sequence with removed insertions may be referred to as an insertion-free MSA object. In some embodiments, the insertion-free MSA object may be used to derive mutation and conservation statistics relative to the consensus sequence.

In some embodiments, conserved regions of the consensus sequence may be determined relative to the variant genome sequences as well as to the genome sequences of the non-variant genomes in the same family as the target genome. In some embodiments, all segments of the consensus sequence that have common corresponding variant segments in the variant genome sequences may be identified and stored. In some embodiments, all segments of the consensus sequence that have common corresponding non-variant or “off-target” segments in the non-variant genome sequences may be identified and stored. In some embodiments, the group of variant segments may be compared with the group of non-variant segments to remove from the group of variant segments any segment that is common to both groups and therefore could lead to a false-positive detection. The remaining group of variant segments may be determined to be the conserved regions of the consensus sequence. In some embodiments, the remaining group of variant segments may undergo filtering based on segment length and/or G+C content, although such filtering may occur later, as discussed below. In some embodiments, each conserved region may be considered for use as a primer.

In some embodiments, the mutation statistics and the conservation statistics may be used to determine properties of inclusivity, such as a primer-set-level inclusivity, discussed below.

In some embodiments, a consensus match matrix (M) may be defined as a binary valued matrix with rows labeled by the genome variants (i), and columns labeled by the positions (j). In some embodiments, an element, mᵢⱼ, in M may be defined by Equation 1, which may be represented as:

$${m}_{ij} = \begin{matrix}\{ 1 if {n}_{ij} = {c}_{j} \\ 0 else\end{matrix} . \qquad Eq . 1$$

In some embodiments, the variable, nᵢⱼ, may be the nucleotide at position j for variant i. In some embodiments, nᵢⱼ may be a deletion. In some embodiments, the variable, cⱼ, may be the nucleotide at position j for the consensus sequence. In some embodiments, insertions may be noted explicitly, by analysis of each variant relative to the consensus sequence, with matrix, I, with numbering similar to M using Equation 2, which may be represented as:

$${I}_{ij} = \{ 1 if variant i opens an insertion at position j 0 else . \qquad Eq . 2$$

In some embodiments, the consensus sequence may be used to identify primers that detect “off-target” genome segments, as noted above. In some embodiments, an “off-target” genome segment may be a genome segment that has a high similarity to the target genome sequence (or to the consensus sequence, if the target genome sequence is not known), however amplification of this “off-target” genome segment may result in a false-positive detection of the target genome sequence. In some embodiments, sequences in the same-family database of sequences may be used to model cross-reactivity, to determine segments that may lead to false-positive detections. In some embodiments, the same-family database may include a plurality of non-variant genome sequences that belong to the same family as the target genome and that, if detected, would result in an erroneous or false-positive detection of the target genome. In some embodiments, cross-reactivity may be measured by a similarity between the consensus sequence to any sequence in the same-family database. In some embodiments, similarity may be measured by global sequence alignment tools. In some embodiments, an EMBOSS Stretcher algorithm may be used for global sequence alignment. In some embodiments, a global sequence alignment procedure may yield the conservation statistics.

In some embodiments of the design system, the mutation statistics for the target genome may determine a degree of similarity between the consensus sequence and each variant genome sequence.

### 2.5 Position-Specific Statistics

Information on positions of conserved regions in the consensus sequence may be used to determine positions of primers for the consensus sequence. In some embodiments, a position of an initial primer selected for a primer set may exclude certain other primers from being selected for the primer set.

In some embodiments, out of all variants, a fraction, fⱼ, of all variants corresponding to matching variants for position j may be defined by Equation 3, where variable Nᵥ represents a total number of variants in the inclusivity database:

$${f}_{j} = {\sum }_{i = 1}^{{N}_{v}} {m}_{ij} / {N}_{v} \qquad Eq . 3$$

In some embodiments, out of all variants, a fraction of all variants corresponding to non-matching variants, gⱼ, for position j may be defined by Equation 4:

$${g}_{j} = 1 - {f}_{j} . \qquad Eq . 4$$

In some embodiments, out of all variants, a fraction of all variants that include at least one insertion, ĝⱼ, may be defined by Equation 5:

$${\hat{g}}_{j} = {g}_{j} + {\sum }_{i = 1}^{{N}_{v}} {I}_{ij} / {N}_{v} \qquad Eq . 5$$

In some embodiments, out of all variants, a set of variants mismatching at position j, Gⱼ, may be defined by Equation 6:

$${G}_{j} = \{ i \vert {m}_{ij} = 0 \} . \qquad Eq . 6$$

In some embodiments, out of all variants, a fraction of all variants corresponding to non-matching variants, gⱼ, may be computed as the number of members in this set (using a “size-of” notation |*|) divided by the total number of variants, as defined by Equation 7, which may be represented as:

$${g}_{j} = \vert {G}_{j} \vert / {N}_{V} . \qquad Eq . 7$$

### 2.6 Primer-Specific Statistics

In some embodiments, primer-specific statistics may be calculated and may be stored in one or more databases. In some embodiments, each primer may be associated with a target variant database storing information relevant to that primer. In some embodiments, a primer may be defined by the 5′ position (p) and 3′ position (q) on the consensus sequence. In some embodiments, the total fraction of mismatches in a primer (gₜₒₜ), may be determined by Equation 8:

$${g}_{tot} ( p , q ) = {\sum }_{j = p}^{q} \vert {G}_{j} \vert / {N}_{V} . \qquad Eq . 8$$

In some embodiments, a unified set of mismatched variants, H (p,q), for the primer may be determined by Equation 9 and Equation 10, where D corresponds to the set of variant indexes for deletions in positions spanned by the primer, and where I corresponds to the set of variant indexes for insertions in positions spanned by the primer:

$${G}_{tot} ( p , q ) = {U}_{j = p , p + 1 , … , q - 1 , q} {G}_{j} , \qquad Eq . 9$$

$$H ( p , q ) = {G}_{tot} ( p , q ) ⋃ D ( p , q ) ⋃ I ( p , q ) . \qquad Eq . 10$$

In some embodiments, the set of mutations, Mᵢ, for variant i may be determined by Equation 11:

$${M}_{i} = \{ j \vert {m}_{ij} = 0 \} . \qquad Eq . 11$$

In some embodiments, the integer set corresponding to the range spanned by the primer, R (p, q), may be represented by Equation 12:

$$R ( p , q ) = \{ p , p + 1 , … , q - 1 , q \} . \qquad Eq . 12$$

In some embodiments, the set of mutations for variant i within the primer, Mᵢ(p,q), may be determined by Equation 13:

$${M}_{i} ( p , q ) = {M}_{i} ⋂ R ( p , q ) \qquad Eq . 13$$

In some embodiments, the set of the variants, W(p,q), with more than one mutation in the primer may be represented by Equation 14:

$$W ( p , q ) = \{ i \vert \vert {M}_{i} ( p , q ) \vert > 1 \} \qquad Eq . 14$$

In some embodiments, mutations near the 3′ positions (q) on the consensus sequence may be taken out of consideration as unusable primer sites because an enzyme that may be essential for a NAAT reaction to occur may initiate its action at the 3′ end of the primer and mutations in this region could prevent the enzyme from functioning. In some embodiments, whether a mutation at position j may be taken out of consideration as unusable may be determined by Equation 15:

$$q - j \leq ε \qquad Eq . 15$$

where ε represents a distance between the 3′-end position (q) and the position of the mutation (j).

In some embodiments, the unified set of sequences taken out of consideration as unusable, H′, which may be stored in the target variant database for the primer, may be calculated using Equation 16:

$${H}^{′} ( p , q ) = {G}_{tot} ( q - ε , q ) ⋃ W ( p , q ) ⋃ D ( p , q ) ⋃ I ( p , q ) \qquad Eq . 16$$

In some embodiments, ε may have a value from 2 to 10. In some embodiments, the value may be in a range from 2 to 5, or 3 to 6, or 4 to 7, or 5 to 8, or 4 to 9, or 5 to 10.

### 2.7 Primer-Set-Level Statistics

In some embodiments of the present technology, primer-set-level statistics may be calculated to define the quantified fraction of mutations for a primer set using two different definitions: a strict definition and a generous definition. In order to calculate the primer-set-level statistics, the following calculations may be performed. In some embodiments, all indexes for a primer set may be represented by P, i.e., the union of all ranges from Equation 12 for all primers of the primer set. In some embodiments, P may be determined by Equation 17:

$$P = {U}_{n} R ( {p}_{n} , {q}_{n} ) . \qquad Eq . 17$$

In some embodiments, all mutations in the primer set, P, for variant i, Mᵢ^{set}, may be calculated using Equation 18:

$${M}_{i}^{set} ( P ) = {M}_{i} ⋂ P = {U}_{n} {M}_{i} ( {p}_{n} , {q}_{n} ) . \qquad Eq . 18$$

In some embodiments, the set of variants with multiple (>k) mutations in the primer set, Wₛₑₜ(P, k), may be represented by Equation 19:

$${W}_{set} ( P , k ) = \{ i \vert \vert {M}_{i} ( P ) \vert > k \} . \qquad Eq . 19$$

In some embodiments, as noted above, to define the quantified fraction of mutations for a primer set, using primer-set-level statistics, two different definitions, one strict and one generous, may be used.

In some embodiments, under the strict definition, a primer set with any mutations in one of the primers, Hₛₑₜ(P), may be represented by Equation 20:

$${H}_{set} ( P ) = {⋃}_{n} H ( {p}_{n} , {q}_{n} ) . \qquad Eq . 20$$

In some embodiments, under the generous definition, a primer set allowing up to k non-fatal mutations in a primer but none fatal, H′ₛₑₜ(P,k), may be represented by Equation 21:

$${H}_{set}^{′} ( P , k ) = {⋃}_{n} {H}^{′} ( {p}_{n} , {q}_{n} ) ⋃ {W}_{set} ( P , k ) . \qquad Eq . 21$$

In some embodiments, the fraction of variants strictly judged to fail to amplify, h (P), may be represented by Equation 22:

$$h ( P ) = \vert {H}_{set} ( P ) \vert / {N}_{V} . \qquad Eq . 22$$

In some embodiments, the fraction of variants generously judged to fail to amplify, h′(P,k), may be represented by Equation 23:

$${h}^{′} ( P , k ) = \vert {H}_{set}^{′} ( P , k ) \vert / {N}_{V} . \qquad Eq . 23$$

In some embodiments, the strict set-level inclusivity, φ′, may be calculated using Equation 24:

$$φ ( P ) = 1 - h ( P ) . \qquad Eq . 24$$

In some embodiments, the generous set-level inclusivity, φ′, (using k=2) may be calculated using Equation 25:

$${φ}^{′} ( P ) = 1 - {h}^{′} ( P , 2 ) . \qquad Eq . 25$$

In some embodiments, a relationship between primer sets under the strict and generous definitions may be represented by Equation 26:

$${H}_{set}^{′} ( P , k ) ⊆ {H}_{set} ( P ) . \qquad Eq . 26$$

In some embodiments, the generous set-level inclusivity, q′, may be greater than or equal to the strict set-level inclusivity, φ, as represented by Equation 27:

$${φ}^{′} ( P ) \geq φ ( P ) . \qquad Eq . 27$$

### 2.8 Cross-Reactivity with Non-Variant, Same-Family “Off-Target” Genomes

In some embodiments, cross-reactivity for off-target genomes globally homologous to the target genome (e.g., non-variant genomes in the same family as the target genome) may be treated differently compared to the treatment of target genomes, discussed above. In some embodiments, each individual off-target genome may be addressed separately and may have different metrics computed for each primer. In some embodiments, matching and mismatching of nucleotides in different parts of the primer may be handled as special cases. In some embodiments, for an oligonucleotide segment between p and q, the number of matching nucleotides may be counted using Equation 28:

$$m ( p , q ) = {\sum }_{j = p}^{q} {m}_{ij} . \qquad Eq . 28$$

In some embodiments, mᵢⱼ is the element in the match matrix from Equation 1 for variant i at position j. In some embodiments, an insertion and/or deletion (“indel”) penalty based on alignment scores may be calculated using Equation 29:

$$π ( p , q ) = {\sum }_{j = p}^{q} {i}_{ij} + {w}_{o} o ( p , q ) + {w}_{l} l ( p , q ) . \qquad Eq . 29$$

In Equation 29, the number of mismatched nucleotides for inserted and deleted nucleotides between p and q is represented by i (p, q), the number of indel-openings is represented by o(p,q) and is multiplied by an indel-opening weight represented by wₒ, and a corresponding number of indel-openings larger than one nucleotide is represented by l(p,q) and is multiplied by a corresponding indel-opening weight represented by wₗ.

In some embodiments, the primer may be distinguished into a critical region (e.g., the last ε nucleotides at the 3′ end) and a non-critical region (e.g., the first δ nucleotides at the 5′ end) may be considered as well, where δ represents a distance from the 5′-end position. In some embodiments, a position-weighted penalty, π′(p, q, ε, δ), may be calculated using Equation 30:

$${π}^{′} ( p , q , ε , δ ) = π ( p + δ , q ) + P ( ε ) . \qquad Eq . 30$$

In Equation 30, P(ε) may be used to strongly penalize the presence of an insertion or a deletion (i.e., an indel) in the last ε nucleotides. In certain embodiments, P(ε) may be 5.0. In some embodiments, the indel correction fraction, c(p, q, ε, δ), for a segment may be calculated using Equation 31:

$$c ( p , q , ε , δ ) = {π}^{′} ( p , q , ε , δ ) / ( q - p + {π}^{′} ( p , q , ε , δ ) ) . \qquad Eq . 31$$

In some embodiments, a generalized matching value, m′(p, q, ε, δ), may be calculated using Equation 32:

$${m}^{′} ( p , q , ε , δ ) = m ( p + δ , q - ε ) + {ω}_{δ} m ( p , p + δ ) + {ω}_{ε} m ( q - ε , q ) . \qquad Eq . 32$$

In Equation 32, ω_{S} and ω_{ε} are weights for the two opposite ends of the primer (non-critical and critical) and may have default values of 0.5 and 2.0, respectively. In some embodiments, a generalized identity fraction, f(p, q, ε, δ), for the primer may be calculated using Equation 33:

$$f ( p , q , ε , δ ) = {m}^{′} ( p , q , ε δ ) / {m}_{max}^{′} ( p , q , ε , δ ) - c ( p , q , ε , δ ) . \qquad Eq . 33$$

In Equation 33, m′ₘₐₓ is the maximum matching and may be calculated using Equation 34:

$${m}_{max}^{′} ( p , q , ε , δ ) = q - p - ε - δ + {ω}_{δ} δ + {ω}_{ε} ε . \qquad Eq . 34$$

In some embodiments, a maximal identity fraction, f′(p,q), is the maximum of the above quantity calculated using Equation 33 across a combined critical and middle region of at least rₘᵢₙ nucleotides (with rₘᵢₙ having a default value of 10) using Equation 35:

$${f}^{′} ( p , q ) = max \{ f ( p , q , 3 , δ ) \vert {δ}_{min} < δ < q - p - {r}_{min} ) \} . \qquad Eq . 35$$

In some embodiments, a value of ε=3 may be used to calculate the maximal identity fraction.

In some embodiments, in the context of set-level same-family conservation, a few individual primers with large identity fractions may be tolerated. However, if a pair of head-to-head oriented primers with a high fraction identity exists, the risk of false amplification may be significant. In some embodiments, for a NAAT that has N primers (denoted P in Equation 17) covering genome positions pₙ to qₙ for n [symbol] S={1, 2, . . . , N} (i.e., the range R in Equation 12), primers cover either the sense strand, S+, or the antisense strand, S., according to Equation 36:

$$S = {S}_{+} ⋃ S , \qquad Eq . 36$$

$$S ⋂ S = ∅ .$$

In some embodiments, the set-level amplicon fraction conservation, φ(P), which may be the minimum of a maximum fraction within each group of sense orientations, may be determined using Equation 37:

$$φ ( P ) = min \{ {max}_{n} ( {f}^{′} ( {p}_{n} , {q}_{n} ) ) , {max}_{m} ( {f}^{′} ( {p}_{m} , {q}_{m} ) ) \vert n ∋ S , m S \} . \qquad Eq . 37$$

In some embodiments, among all genomes in the same-family database of off-target sequences, the group-level fraction, φ′(P), may be defined as the maximum across all set-level generalized fractions, φᵢ, as represented by Equation 38:

$${φ}^{′} ( P ) = {max}_{i} ( {φ}_{i} ( P ) ) . \qquad Eq . 38$$

### 2.9 Cross-Reactivity with Genomes of Off-Target Background Substances

In some embodiments, a candidate single primer may be compared to a set of genome sequences for background substances that might be present in sample specimens. In some embodiments, to provide broad coverage, the candidate single primers may be compared to sequences in an off-target database of representative genomes of related organisms (e.g., non-variants in the same family as the target genome) as well as unrelated background substances. In some embodiments, the cross-reactivity may be calculated by a BLAST procedure, which is a publicly available procedure accessible at https://blast.ncbi.nlm.nih.gov/Blast.cgi. In some embodiments, a candidate single primer may be subjected to a BLASTN procedure to search against the background database under standard parameters for a local alignment BLASTN-short task. In some embodiments, the local cross-reactivity score, f (p,q), for a variant genome and query sequence between positions p and q may be calculated using Equation 39:

$$f ( p , q ) = I ( A ) ( k - h ) / ( q - p ) . \qquad Eq . 39$$

In Equation 39, I(A) may be the fraction identity within an aligned gap-free hit segment, A, between positions h and k, and k-h and q-p may be the lengths of the alignment and query segments, respectively. The gap-free hit region, A, may identified by BLASTN to maximize I(A).

In some embodiments, at the primer-set level, the fraction, q, within a suspected “amplicon” pair may be defined as the maximum pair of fractions provided by a hit from each group of sense orientations satisfying a threshold for the hit genome positional separation, Δₘₐₓ, and may be calculated using Equation 40:

$$Eq . 40$$

$$φ ( P ) = {max}_{m , n} \{ min ( f ( {p}_{n} , {q}_{n} ) , f ( {p}_{m} , {q}_{m} ) ) \vert n ∈ S , m ∈ S\_ , {h}_{m} - {k}_{n} < {Δ}_{max} \} .$$

In Equation 40, the quantity f (pᵣ, qᵣ) may be determined from the expression in Equation 41:

$$f ( {p}_{r} , {q}_{r} ) = I ( {A}_{r} ) ( {k}_{r} - {h}_{r} ) / ( {q}_{r} - {p}_{r} ) \qquad Eq . 41$$

$$for$$

$$r = n , m .$$

In some embodiments, primer sets may be filtered to have a maximum value for φ(P). For example, in some embodiments, φ(P) may be set to no greater than 0.8.

### 3. Screening of Primers and Primer Sets

### 3.1 Screening of Initial Primers to Determine Candidate Primers

According to some embodiments of the design system of the present technology, the consensus sequence, the mutation statistics, and the conservation statistics, described above, may be used to determine a plurality of candidate single primers for the consensus sequence. The plurality of candidate single primers may, in some instances, be considered a curated database of potential primers.

In some embodiments, the consensus sequence may be used to identify initial single primers, which may include all potential single primers at all genome positions on the target genome (as represented by the consensus sequence). Each of the initial single primers determined from the consensus sequence may be vetted for its suitability to be included in a new test primer set for the target genome corresponding to the consensus sequence. In some embodiments, initial single primers corresponding to certain genome positions within some genome regions of the consensus sequence may be excluded from being candidate single primers due to cross-reactivity to background and non-variant genomes, to avoid or reduce the occurrence of false-positive detections, as described above. In some embodiments, some but not all of the initial single primers determined from the consensus sequence may be vetted. For example, 50% or 60% or 70% or 80% or 90% of the initial single primers may be vetted.

In some embodiments of the present technology, each of the initial single primers of the consensus sequence may be vetted to ensure a particular complexity or primer length, i.e., a number of nucleotides in the primer. In some embodiments, vetting for a desired complexity may take into considering a desired degree of detection specificity, which would favor longer primer lengths, and also may take into consideration a desire to have detections occur quickly, which would favor shorter primer lengths, to arrive at a range of primer lengths for the candidate single primers. In some embodiments, the range may be 5 to 40 nucleotides, or 10 to 35 nucleotides, or 15 to 30 nucleotides, or 15 to 27 nucleotides. In some embodiments, initial single primers having a number of nucleotides outside of the range may be excluded from being candidate single primers.

In some embodiments of the design system, each of the initial single primers of the consensus sequence remaining after cross-reactivity vetting and after complexity vetting may undergo oligo screening. In oligo screening, a primer's kinetic and thermodynamic properties may be determined and values for these properties may be stored for later use. In some embodiments, such oligo characteristics of the primer may enable a determination of hybridization kinetics between the primer and the target genome. In some embodiments, the kinetic and thermodynamic properties of the primer may be determined from the primer's sequence of oligonucleotides.

In some embodiments, the kinetic and thermodynamic properties determined in oligo screening of a primer may include any one or any combination of: an average linguistic sequence complexity of the primer; a melting point (Tₘ) of the primer; a percentage of guanine (G) and cytosine (C) nucleotides in the primer; a Gibbs free energy (ΔG) for polymerase initiation of the primer; an enthalpy (ΔH) associated with a reaction involving the primer; an entropy (ΔS) associated with a reaction involving the primer; a Gibbs free energy (ΔG) for terminal (“end cap”) nucleotides of a duplex molecule formed of the primer and its complementary counterpart; thermodynamic properties (e.g., ΔG, ΔH, ΔS) of the duplex molecule computed based on next nearest neighbor models; a melting point (Tₘ) of the duplex molecule computed based on known algorithms (e.g., PrimerExplorer); a probability of base-pair hybridization for the primer (e.g., determined using a reaction partition function); etc. As will be appreciated, the foregoing list is not an exhaustive list of properties that may be determined in oligo screening but instead are examples of properties that may be considered in oligo screening.

In some embodiments, a primer may be evaluated according to an analytical manipulation of a property of the primer. In some embodiments, a quadratic difference such as shown in Equation 42 may be used to evaluate a primer:

$${f}_{2} ( x , v ) = {( x - v )}^{2} . \qquad Eq . 42$$

In Equation 42, x represents a value for a property (e.g., Tₘ) of the primer and v represents an optimal value for that property.

In some embodiments, a primer may be evaluated for its kinetic properties for on- and off-pathway reactions. Off-pathway reactions may be detrimental to an intended amplification reaction by possibly amplifying side-products that may lead to false-positive detections. In some embodiments, the kinetics for on- and off-pathway reactions may be simulated using NUPACK, which is publicly available software accessible at http://www.nupack.org/. In some embodiments the kinetics for on- and off-pathway reactions may be simulated using RNAstructure, which is publicly available software accessible at https://rna.urmc.rochester.edu/RNAstructure.html. In some embodiments, NUPACK and/or RNAstructure may be used to generate energetic properties (e.g., AGs) for side-product formation and/or formation of hairpin structures of the primer and/or formation of homodimers (symmetric self-complementary structures) of the primer. In some embodiments, a property determined for a primer during oligo screening may cause the primer to be excluded from being a candidate single primer. For example, if a primer is determined to have a Tₘ that is excessively high, the primer may be excluded from being a candidate single primer. In some other embodiments, a primer may not be excluded from being a candidate single primer based on a particular property determined from the primer because second-order effects, such as when the primer is paired with another primer, may cause the pair of primers to have advantageous properties that would not be possible with each primer by itself; thus, the primer may be included as a candidate single primer.

In some embodiments, properties determined from oligo screening as well as other screening of a primer may be used in computing a fitness score of a new test primer set comprised of the primer. For example, the oligo-screening properties determined for the primer may be used as values for a multi-variable scoring function that simultaneously takes into consideration any two or more properties derived from oligo sequences of the new test primer set, as described in more detail below.

### 3.2 Primer-Pair Properties

In some embodiments of the design system of the present technology, building of a primer set may take into account properties of primer pairs. A property of a single primer may in some cases be adversely affected when paired with another primer (e.g., primer A) in a primer set, whereas in some other cases that single primer may be advantageously affected when paired with a different primer (e.g., primer B). In some embodiments, primer pairs may be evaluated, and properties of the primer pairs may be stored for use in future computations and evaluations. Examples of primer-pair properties that may be taken into account may include any one or any combination of: a linear combination of individual properties of each of the primers of the pair (e.g., a difference in melting points (ΔTₘ) of the two primers of the pair); quadratic differences between properties of each of the primers of the pair; free energies of formation (AGs) for hetero-dimer formation involving the primers of the pair; a genomic positional separation between the two primers of the pair; and for LAMP-based reactions where FIP and BIP primers are involved, free energies of formation (AGs) for hairpin and homo-dimer formation for both the FIP and BIP primers.

### 3.3 Primer-Set Properties

In some embodiments of the present technology, a primer set may be evaluated based on its full set of primers, i.e., all the primers of the primer set. Properties or features of the full primer set may be referred to as set-level properties or features. In some embodiments, set-level properties or features that may be taken into consideration in evaluating a primer set may include any one or any combination of:
An average of some or all primer features for a specific property (e.g., an average length of all primers in the primer set, an average G+C content);
A Tₘ-high/low difference, which may be particularly relevant to LAMP-based reactions. In some embodiments, this difference may correspond to the difference, F_{HL}, between a sum of melting points of all highest-Tₘ primers of the primer set and a sum of melting points of all lowest-Tₘ primers of the primer set. This difference may be calculated according to Equation 43:

$${F}_{HL} = {\sum }_{i ∈ H} {Tm}_{i} - {\sum }_{j ∈ L} {Tm}_{j} . \qquad Eq . 43$$

In Equation 43, H may represent high-Tₘ inner primers (e.g., F1c/B1c, LF/LB), and L may represent low-Tₘ outer primers (e.g., F2/B2, F3/B3);
A Tₘ root-mean-square deviation average (rmsd-Tₘ difference average), which may be particularly relevant to LAMP-based reactions. The rmsd-Tₘ difference average may correspond to all symmetry-related LAMP primer pairs (F1c-B1c, F2-B2, etc.), and may be calculated according to Equation 44:

$$1 / N \sqrt{{\sum }_{j ∈ i}^{N} {( Tm ( {F}_{i} ) - Tm ( {B}_{i} ) )}^{2}} . \qquad Eq . 44$$

A difference in free energies of formation (AG) for hetero-dimer formation between FIP and BIP, which may be particularly relevant to LAMP-based reactions; and
Differences in free energies of formation (AG) between side-product formation of some or all known side-products relative to target reaction products.

In some embodiments of the present technology, set-level properties of a primer set may be computed for a continuous span of nucleotides encompassing all primers of the primer set, including nucleotides bridging a pair of primers. For example, an average G+C content may be computed for only the full set of primers for the primer set or may be computed for the continuous span of nucleotides encompassing the full set of primers.

### 4. Determination of Primer Sets

### 4.1 Introduction

According to some embodiments of the present technology, the design system may generate a single set of primers for detecting a target genome, or may generate a plurality of sets of primers for detecting the target genome. As noted above, the design system may use a multi-variable function configured to determine a fitness score that may be used to predict a primer set's suitability or acceptability for detecting the target genome.

According to some embodiments, the design system may derive a collection of acceptable sets of primers from an initial primer set comprised of a plurality of candidate single primers selected for the initial primer set, or may derive the collection of acceptable sets of primers from a modified primer set that may be a modification of a previously derived primer set (e.g., a modification of the initial primer set). In the latter case, the modified primer set may be referred to as a child primer set, and the previously derived primer set may be referred to as a parent primer set. In some embodiments, the collection of acceptable sets of primers may be derived in parallel from a plurality of different initial primer sets, with each initial primer set being comprised of candidate single primers selected for that initial primer set.

In some embodiments, an initial primer set may be built by randomly selecting an initial primer and selecting other primers one by one, based on the initial primer and any other primer previously selected to be a member of the initial primer set. In some embodiments, an initial primer set may be built by selecting an initial primer based on one or more predetermined characteristics (e.g., one or more oligo characteristics) and selecting other primers one by one, based on the initial primer and any other primer previously selected to be a member of the initial primer set.

In some embodiments, a modified (“child”) primer set may be built by modifying a previously derived (“parent”) primer set. Modification of the parent primer set may be comprised of modifying at least one primer of the parent primer set. For example, the parent primer set may be modified by combining one or more primers of the parent primer set with one or more primers of a second parent primer set (i.e., another previously derived primer set), and/or by changing a position of a starting point or an ending point of at least one primer of the parent primer set, and/or by causing a mutation in at least one primer of the parent primer set, and/or by swapping a primer of the parent primer set with another primer from a collection of candidate single primers, etc. In some embodiments, a child primer set may be derived as a combination of two parent primer sets, in which a group of single primers from one of the parent primer sets and a group of single primers from the other of the parent primer sets is included in mixed order in the child primer set.

### 4.2 Building an Initial Primer Set from an Initial Primer

In some embodiments of the design system, an initial primer set may be referred to as a new test primer set and may be built from an initial primer that is selected randomly from a set of candidate primers (e.g., the set of candidate single primers discussed above). Subsequently, other primers may be added one by one at suitable positions relative to previously selected primers, until a full primer set with acceptable properties is obtained. For example, a second primer may be chosen based on, among other things, a desired separation distance from the initial primer and/or a desired genome position, and a third primer may be chosen based on, among other things, desired separation distances between the first primer and the second primer and/or a desired genome position. In some embodiments, a desired number of primers may be predetermined based on kinetic considerations (e.g., a desired testing time for a NAAT).

In some embodiments, the design system may build a plurality of initial primer sets in a parallel. For example, a plurality of CPUs may each be programmed to randomly select an initial primer and to build a new test primer set by adding primers one by one, as discussed above. In this way, multiple different initial primer sets may be generated in a relatively short amount of time (e.g., in the same amount of time as for generating one initial primer set).

In some embodiments, each new test primer set may have an optimal analytical performance based on its constituent primers, as reflected in a calculated value for a target function (e.g., the performance target function 120) for that primer set. In some embodiments, the optimal analytical performance may correspond to a lowest value for the target function. In some embodiments, as a new test primer set is being built, step-wise interim values of the target function may be determined for each primer selected for the primer set.

### 4.3 Target Function

In some embodiments, a target function, T (P, L), may have a value that is computed from a selection of individual features, F, which may be computed from the primer-set specifications P and L for a given genome sequence (e.g., the target genome's nucleic-acid sequence). In some embodiments, each feature, F, may represent a property for one or more primers. In some embodiments, a primer set may be represented by 2*N integers, where the primer set has N sub-primer 5′ starting positions, P, on the genome, and N primer lengths, L. Equation 45 shows representations for P and L:

$$P = ( {p}_{1} , {p}_{2} , … , {p}_{N} ) , \qquad Eq . 45$$

$$L = ( {l}_{1} , {l}_{2} , … , {l}_{N} ) .$$

As a non-limiting, illustrative example, N may be 8 for a LAMP-based NAAT. In some embodiments, a primer, j, may span positions pⱼ to pⱼ+lⱼ.

In some embodiments, the target function may be a linear target function, T (P, L), which may be represented by a linear model according to Equation 46:

$$Eq . 46$$

$$T ( P , L ) = {\sum }_{i = 1}^{{n}_{S}} {\sum }_{i , j = 1}^{N} {w}_{ij} {F}_{j}^{i} ( {p}_{j} , {l}_{j} ) + {\sum }_{i = 1}^{{n}_{P}} {\sum }_{j = 1}^{N} {\sum }_{k = j + 1}^{N} {w}_{ijk} {F}_{j , k}^{i} ( {p}_{j} , {l}_{j} , {p}_{k} , {l}_{k} ) + {\sum }_{i = 1}^{{n}_{Z}} {w}_{i} {F}_{set}^{i} ( P , L ) + c$$

In Equation 46, Fⱼⁱ (pⱼ, lⱼ) in the uppermost term may represent an i^{th} feature for primer j, the middle term may represent features for primer pairs (e.g., primer j and primer k), and the lowermost term may represent features calculated for all primers in the primer set being built, which may have fewer primers than a full primer set. In Equation 46, wᵢ, wᵢⱼ, and wᵢⱼₖ, each represent a linear weight for its respective term.

In Equation 46, sub-totals of different features may be computed at a single-primer level, n_{S}, a primer-pair level, n_{P}, and a primer-set level, n_{Z}. A total number of features, nₜₒₜₐₗ, may be calculated using Equation 47:

$${n}_{total} = {Nn}_{S} + N / 2 ( N - 1 ) {n}_{P} + {n}_{Z} \qquad Eq . 47$$

In some embodiments, the target function may be a non-linear target function, U (P,L). In some embodiments, instead of linear weights, each level may be weighted according to a univariate function, u. For example, the single-primer level of the target function, U(P,L), may be represented by Equation 48:

$${U}_{S} ( P , L ) = {\sum }_{i , j} {u}_{ij} ( {F}_{j}^{i} ( {p}_{j} , {l}_{j} ) ) . \qquad Eq . 48$$

In some embodiments, each univariate function may have an optimal weight that may be determined by training, in which correlations between features and their respective values may be determined by experimentation and correlation refinement, such as is typical for classical machine-learning techniques. In some embodiments, techniques such as PLS (partial least squares) regression may be used to find optimal weights.

### 4.4 Incremental Addition of a Primer

As noted above, an initial primer may be built by selecting each primer one by one, after an initial primer is randomly selected. In some embodiments of the present technology, an optimal next primer may be added based on minimizing a combined sum of changes. In such an analysis, k−1 previously selected or “first” primers may have already been included in an incomplete primer set, and a kth primer may be added. In some embodiments, an increase in the target function, ΔₖT (pₖ,lₖ), may be estimated according to Equation 49:

$$\begin{matrix}{Δ}_{k} T ( {p}_{k} , {l}_{k} ) = {\sum }_{i}^{{n}_{S}} & {w}_{i k} {F}_{k}^{i} ( {p}_{k} , {l}_{k} ) + {\sum }_{i}^{{n}_{P}} & {\sum }_{j = 1}^{k - 1} {w}_{i j k} {F}_{j , k}^{i} ( {p}_{j} , {l}_{j} , {p}_{k} , {l}_{k} ) .\end{matrix} \qquad Eq . 49$$

The expression in Equation 49 relates to a linear target function; as will be appreciated, univariate functions may be used for a non-linear target function. In Equation 49, the single-primer-level term on the left may have pre-computed values stored in a memory device, whereas the primer-pair-level term on the right may be computed on-the-fly. The optimal next primer may be the primer that minimizes the combined sum of changes of Equation 49. In this analysis, the total number of features to be computed may be determined by Equation 50:

$${n}_{step} = ( k - 1 ) {n}_{P} . \qquad Eq . 50$$

In some embodiments, because nₛₜₑₚ<<nₜₒₜₐₗ, the relatively fewer number of features can be computed in a reasonable time to enable a tractable search to be performed for incomplete primer sets. Examples of different algorithms that may be used for this purpose include, but are not limited to:
1. An algorithm to minimize ΔₖT(pₖ, lₖ) from an exhaustive search within reasonable search ranges for pₖ, lₖ. Such an algorithm can be computed faster if only pre-computed single-primer terms (and possibly only a few of primer-pair features) are used;
2. An algorithm to minimize ΔₖT(pₖ, lₖ) from a sub-sampled search within reasonable search ranges for pₖ, lₖ; and
3. An algorithm to compute ΔₖT(pₖ, lₖ) from a sub-sampled search within reasonable search ranges for pₖ, lₖ, in which computations are aborted when acceptable values are obtained. This algorithm can be performed via a randomized search or via deterministic incrementation of positional primer specifications.

### 4.5 Analytical Performance, Inclusivity, and Cross Reactivity

An optimal clinical performance of a NAAT may require a good analytical performance (e.g., a quick time to detection, an ability to detect a small amount of a target genome), a high inclusivity (e.g., an ability to detect the target genome and most if not all of its known variants), and minimal cross-reactivity (e.g., an acceptably small rate of false-positive detections). In some embodiments, an optimal clinical performance may be achieved by a combined performance function, T_{comb}, which may be computed according to Equation 51:

$${T}_{c o m b} ( P , L ) = T ( P , L ) + s ( 1 - {φ}_{i n c l}^{′} ( P , L ) , {w}_{i n c l} , 1 - {I}_{max} ) + s ( {φ}_{C R}^{′} ( P , L ) , {w}_{CR} , {C}_{max} ) . \qquad Eq . 51$$

In Equation 51, s is a penalty function, which may be defined according to Equation 52:

$$s ( x , w , m ) = 0 if x < m {and ( ( x - m ) / w )}^{2} else . \qquad Eq . 52$$

In Equation 51, T is the target for analytical performance (e.g., see Equation 46), and φ′_{incl} and φ′_{CR} are the inclusivity and same-family cross-reactivity, respectively (see Equations 25 and 38). In Equation 51, Iₘᵢₙ and Cₘₐₓ are the minimum and maximum thresholds, and w_{incl} and w_{CR} are weights for the these thresholds, respectively.

### 5. Optimization, Filtering, and Diversification of Primer Sets

### 5.1 Introduction

The collection of acceptable primer sets generated in the previous steps, discussed above, contain primer sets with acceptable individual properties and local optimum properties. In some embodiments, the collection may be refined by a global optimization procedure to determine a global optimum, in which some or all interactions between a larger number of primer sets may be calculated and evaluated. In some embodiments, the global optimization procedure may ensure that the primers in the primer set have been evaluated for second-order or higher-order effects between the primers, including effects between more than two of the primers. The global optimization procedure may be comprised of a stochastic optimization method that applies a genetic algorithm (GA), discussed below, to determine optimized primer sets, which may undergo filtering and culling to arrive at a diverse group of primer sets for detecting the target genome.

### 5.2 Optimization Using a Genetic Algorithm (GA)

In some embodiments of the design system of the present technology, optimization of a primer set may be performed on a new primer set (e.g., an initial primer set, discussed above) or on a previously derived sub-optimal primer set, which may be a primer set from a database of sub-optimal primer sets stored in a memory device. The sub-optimal primer set may be derived as discussed above, and may be comprised of high-quality primers selected to optimize a target function. The sub-optimal primer set may be “sub-optimal” in the sense that it has not been subjected to optimization. As noted above, in some embodiments, optimization may determine a global optimum using a GA that accounts for higher-order effects between a larger number of primers (e.g., more than two primers) of the sub-optimal primer set. In some embodiments, the GA may be comprised of a metaheuristic search algorithm that derives a population of candidate primer sets through operations that may mimic biological processes of mutation and crossover. In some embodiments, the GA may mimic a mutation as a random permutation of a starting position of one of the primers by changing the primer's length (e.g., by +/−1 or another small number). In some embodiments, the GA may mimic a crossover to form a child primer set by mixing primer positions and primer lengths of two parent primer sets, with the child primer set inheriting specifications from either of the two parent primer sets. In some embodiments, the GA may depend on several probabilistic choices and therefore the GA may be stochastic, such that different results may be obtained when GA-optimization is performed on the same primer multiple times. In some embodiments, the GA may be performed iteratively in successive generations, with results from the various generations being accumulated or with results of each successive generation being based on results from at least one previous generation. In some embodiments, the GA may be run for a finite number of generations. In some embodiments, the finite number of generations may be 5, or 10, or 15, or 20, or 30, or 50, or 100.

In some embodiments, the GA may be comprised of a first-generation procedure in which at least one “parent” primer set is selected from a collection of previously derived parent primer sets, and a fitness score for each selected parent primer set is obtained. In some embodiments, the collection of parent primer sets may be stored in a database along with the fitness scores of the parent primer sets. As discussed above, a primer set's fitness score may be a predicted performance score of the primer set. In some embodiments, the parent primer set may be selected according to its rank in the collection of parent primer sets. For example, the collection of parent primer sets may be sorted according to fitness score, with each parent primer set having a rank based on its fitness score. In some embodiments, a parent primer set of the collection may be selected if the parent primer set has a rank above a threshold (e.g., top 50% of all the fitness scores of the collection).

In some embodiments, the GA may derive a new or “child” primer set from the parent primer set by performing a mutation operation and/or a crossover operation, such as discussed above. In some embodiments, the operation(s) performed may be chosen randomly. The mutation operation may use one parent primer set and the crossover operation may use two parent primer sets.

In some embodiments, during GA-optimization of a parent primer set, each child primer set derived from the parent primer set may be added to a collection of child primer sets for that parent primer set. The collection of child primer sets may include fitness scores determined for the child primer sets. In some embodiments, the collection of child primer sets may be stored in a database in a memory device. In some embodiments, during a growth phase of the GA-optimization, the collection may grow until the collection contains a desired number of child primer sets. In some embodiments, the optimized primer set for the parent primer set may be the primer set having the best fitness score amongst the fitness scores of the child primer sets in the collection and the fitness score of the parent primer set. In some embodiments, a group of the child primer sets having the highest fitness scores (e.g., the top 10% of fitness scores) may be designated a collection of optimized primer sets for the parent primer set. The parent primer set may be included in the collection of optimized primer sets, if the parent primer set's fitness score is within the range of the highest fitness scores. In some embodiments, the optimized primer set(s) may be stored and/or output for use in a diagnostic test (e.g., a NAAT) for detecting the presence of the target genome in a sample.

In some embodiments, during GA-optimization of a parent primer set, a child primer set may be added to a collection of child primer sets only if a fitness score determined for the child primer set meets one or more requirements. For example, if the child primer set's fitness score is better than that that of the parent primer set, the child primer set may be added to the collection of child primer sets. In some embodiments, during a growth phase of the GA-optimization, the collection may grow until the collection contains a desired number of child primer sets or until a predetermined number of child primer sets have been derived from the parent primer set. In some embodiments, the optimized primer set for the parent primer set may be the primer set having the best fitness score amongst the fitness scores of the child primer sets in the collection and the fitness score of the parent primer set. As in the case above, in some embodiments, a group of the child primer sets having the highest fitness scores (e.g., the top 10% of fitness scores) may be designated a collection of optimized primer sets for the parent primer set. The parent primer set may be included in the collection of optimized primer sets, if the parent primer set's fitness score is within the range of the highest fitness scores. In some embodiments, the optimized primer set(s) may be stored and/or output for use in a diagnostic test (e.g., a NAAT) for detecting the presence of the target genome in a sample.

In some embodiments, during GA-optimization of a parent primer set, a child primer set may replace the parent primer set if a fitness score for the child primer set is better than that of the parent primer set. If the fitness score for the child primer set is not better than that of the parent primer set, the child primer set may be discarded and, in a next iteration of the GA-optimization, a new child primer set may be derived from the parent primer set. On the other hand, if the parent primer set is replaced by the child primer set, the parent primer set may be discarded and, in a next iteration of the GA-optimization, a second-child primer set may be derived from the child primer set. In some embodiments, GA-optimization may proceed for a predetermined number of iterations, with the primer set remaining after all the iterations being the optimized primer set for the parent primer set.

In some embodiments, GA-optimization of a parent primer set may utilize a predetermined criterion. Whether a child primer set is added to a collection of child primer sets (the population) or replaces the parent primer set may be determined by comparing the child primer set's fitness score, f_{C}, with the parent primer set's fitness score, f_{P}, using a soft Boltzmann criterion. In some embodiments, f_{C} is better than f_{P} if f_{C}<f_{P} or if the expression in Equation 53 is met:

$$\begin{matrix}{e}^{( {f}_{P} - {f}_{C} ) / T} > r , & r ∼ U ( 0 , 1 )\end{matrix} . \qquad Eq . 53$$

In Equation 53, r is a random number and may be drawn from a uniform distribution between 0 and 1, and T is a population temperature of the collection of child primer sets, where lower temperatures may correspond to higher selection pressures. If the temperature, T, is high the likelihood is higher for adding new members to the population, whereas in contrast, low temperatures would enforce to only keep the very fittest individuals in the population (referred to here as “selection pressure”). In some embodiments, T may be variable and may be decreased adaptively throughout the iterations (generations) by setting T to 0.5 times the standard deviation among the fitness scores for the best-ranked half of the population of the collection. In some embodiments, a fixed number of computations of fitness scores of child primer sets may be performed, enabling the population to increase in size. In some embodiments, the population may be culled to a fixed size based on fitness score and a similarity between two or more primer sets. In some embodiments, the similarity may be calculated according to Equations 54-58, discussed below.

Two primer sets P and Q may be denoted as shown in Equation 54:

$$P = ( {p}_{1} , {p}_{2} , … , {p}_{n} ) and Q = ( {q}_{1} , {q}_{2} , … , {q}_{n} ) . \qquad Eq . 54$$

The similarity between P and Q may be based on a positional distance between P and Q, d (P, Q), which may have 3′- and 5′-end positions. In Equation 54, the first n/2 numbers may represent the 3′ positions and the last n/2 numbers may represent the corresponding 5′ positions. In some embodiments, d may be calculated using Equation 55:

$$\begin{matrix}d ( P , Q ) = \frac{1}{n} {\sum }_{i = 1}^{n} & ln ( \vert {p}_{i} - {q}_{i} \vert + 1 )\end{matrix} . \qquad Eq . 55$$

An average distance from each individual primer k, dx, may be calculated using reciprocal weighting to put emphasis on short distances, as given in Equation 56:

$${d}_{k} = \frac{n}{( \begin{matrix}{\sum }_{i = 1}^{n} & ( \frac{1}{d ( i , k ) + 0.1} ) )\end{matrix}} . \qquad Eq . 56$$

In Equation 56, i is an index that loops through all individual child primer sets in the population of the collection of child primer sets.

In some embodiments, a diversity score, sₖ, may be calculated as a weighted sum of the fitness score, fₖ, and the average distance, dₖ, using Equation 57:

$${s}_{k} = {w}_{f} {f}_{k} - {w}_{d} {d}_{k} . \qquad Eq . 57$$

In Equation 57, w_{f} and w_{d} may be weights defined by Equation 58:

$${w}_{f} = \frac{2}{T} and {w}_{d} = \frac{3}{σ} . \qquad Eq . 58$$

In Equation 58, T is the temperature and σ is the standard deviation among all average distances (i.e., among all dₖ).

In some embodiments, at the end of an iteration or generation, of all the child primer sets derived for the collection, a fixed number of child primer sets (e.g., the ones having the best possible diversity scores) may be preserved for the next iteration or generation. In some embodiments, after a fixed number of iterations or generations, GA-optimization of a parent primer set may terminate. In some embodiments, upon termination of the GA-optimization of the parent primer set, a fixed number of the child primer sets with the highest fitness scores may remain in the collection of child primer sets, with the other, low-scoring child primer sets being discarded. In some embodiments, a diverse group of the remaining child primer sets, each member of the group being relatively diverse from other members of the group, may be retained and may be designated a collection of optimized primer sets for the parent primer set that underwent optimization using the GA. In some embodiments, the GA may store the collection of optimized primer sets in a memory device and/or may output the collection for use in detection tests (e.g., NAATs) requiring amplification of the nucleic acid of the target genome for which the optimized primer sets were produced.

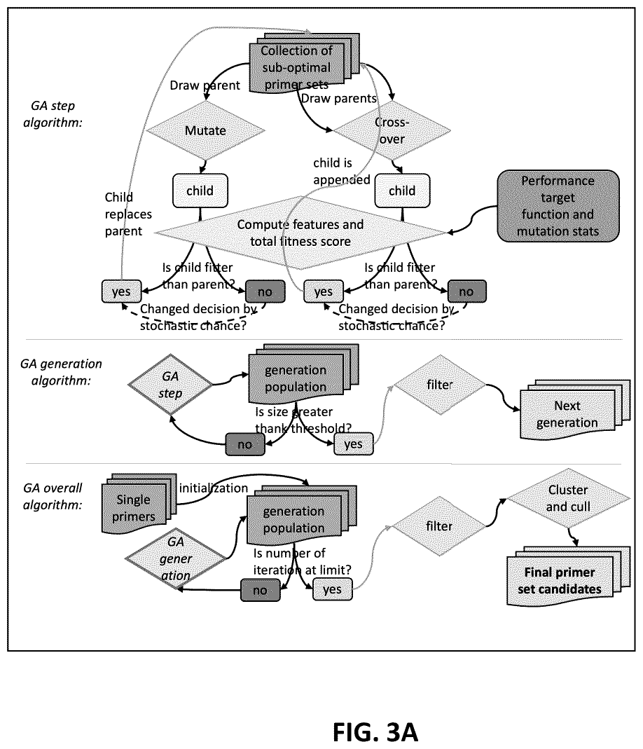

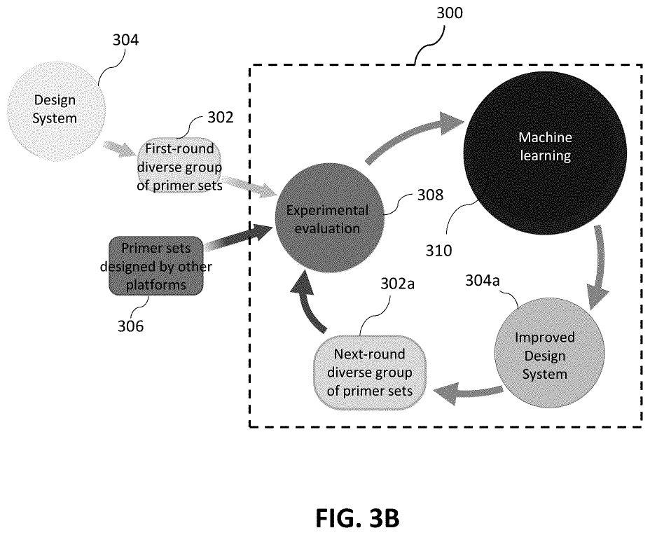

FIG. 3A shows a flow diagram summarizing the procedures of the GA discussed above. Although the procedures discussed above for optimization of a parent primer set using the GA describe the optimization of one parent primer set, it should be understood that optimization of a plurality of different parent primer sets may be performed in parallel, in some embodiments. Further, in some embodiments, due to the stochastic nature of optimization using the GA, the same parent primer set may be optimized a plurality of times using the GA, with each optimization yielding a different collection of one or more optimized primer sets. Furthermore, during optimization of a parent primer set, parallel processing may be performed such that a plurality of different child primer sets may be derived simultaneously or nearly simultaneously for the parent primer set.

### 5.3 Filtering to Produce Filtered Primer Sets

In some embodiments of the present technology, the collection of optimized primer sets may be filtered against homology with off-target nucleic-acid sequences corresponding to background substances that may be found in samples containing the pathogen or target genome to be detected. The filtering may eliminate primer sets that may be susceptible to detecting a background substance, which may lead to a false-positive detection for the target genome. In some embodiments, off-target background substances may be comprised of a variety of known genomic materials (e.g., microbial substances) present in humans and likely to be found in samples obtained from humans. In some embodiments, nucleic-acid sequence homology of each primer in an optimized primer set may be compared to some or all of such known genomic materials. As discussed above, sequence homology may be estimated using BLASTN, with default settings for short local alignments. In some embodiments, if two or more primers have high local homology to both strands located within a relatively short segment on the same background-substance genome, there may be an unacceptable risk of false amplification of this background-substance genome. In some embodiments, the two or more primers with high local sequence homology may be filtered to remove all primer sets having above a maximum local-sequence-homology fraction between any pair of such genomic sequences. In some embodiments, primer sets having 80% homology and a separation of 1000 nucleotides may be filtered. In some embodiments, primer sets having 60% or 70% or 90% homology and a separation of 600 or 700 or 800 or 900 or 1100 or 1200 nucleotides may be filtered.

### 5.4 Clustering and Culling to Produce Diverse Primer Sets

Diversity of primer sets may be beneficial for situations where speed and efficiency are important for determining a best primer set for detecting a pathogen of interest (“best” meaning the best amongst the primer sets being studied). In some embodiments, after the optimized primer sets have been filtered to remove primer sets that may be susceptible or prone to detecting a background substance, diversification of the remaining optimized primer sets may occur. In some embodiments, the remaining optimized primer sets may be clustered into groups of similar primer sets and culled to remove all but one primer set (or all but a few primer sets) from each of the groups. In this way, the optimized and diversified primer sets may lead to a more efficient determination of the best primer set. For instance, a group of similar primer sets may contain primer sets that are highly similar to each other and therefore may not yield additional information because the similar primer sets may behave similarly to each other. That is, when similar primer sets are used in clinical tests to determine the best primer set for real-world detections of a pathogen of interest, each of the similar primer sets may provide information that is largely similar to information provided by the other similar primer sets, and therefore the additional testing may amount to spending a great deal of time and resources to obtain little to no additional information. In some embodiments, clustering may group together redundant primer sets and/or primer sets that differ from each other in insignificant ways. For example, within a group of similar primer sets, one primer set may differ from another primer set by less than a predetermined number of nucleotide insertions or deletions. As will be appreciated, other parameters may be used to determine similarity for a particular group. Each group of similar primer sets may differ from other groups of similar primer sets such that the groups are diverse from each other. Therefore, by culling the groups so that one or a few representative primer sets remain for each group, a collection of diverse primer sets remain and may have a higher probability of yielding useful information from a smaller number of primer sets. As noted above, when diverse primer sets are used in clinical tests, the likelihood that they behave similarly to each other to detect a pathogen of interest is relatively lower, and therefore the diverse primer sets may yield more useful information than primer sets that are similar to each other.

In some embodiments, clustering may be performed based on average distance, dₖ, as determined using Equation 56. In some embodiments, known clustering algorithms may be used (e.g., DBscan, which is publicly available from scikit-learn). In some embodiments, a cut-off for cluster similarity (or number of groups) may be used. For example, a cut-off may be approximately 10 (or 5 or 15 or 20 or 25) clusters for each population of 250 (or 200, or 300 or 400 or 500 or 1000) optimal primer sets. In some embodiments, two or three members for each cluster may be kept as representative candidates. In some embodiments, the two or three members may have the highest fitness scores of the cluster. In some embodiments, if a cluster has fewer than three members then all members are kept. In some embodiments, multiple rounds of primer design and optimization may be run repeatedly (simultaneously and/or sequentially), with results produced from the multiple rounds pooled into a primer-set-candidate poll. In some embodiments, the primer-set-candidate poll may be clustered and culled together to produce a larger collection of diverse primer sets.

FIG. 3B shows a flow diagram of a virtuous cycle 300 for designing primer sets, according to some embodiments of the present technology. In some embodiments, virtuous cycle 300 may apply experimental results and analyses of clinical tests to machine learning to refine the design system discussed above. In some embodiments, the virtuous cycle 300 may be used to modify parameters and/or mathematical relationships involved in calculating the fitness score. In some embodiments, the virtuous cycle 300 may be used to modify the GA, including modifying parameters and/or mathematical relationships involved in any one or any combination of Equation 1 through Equation 58 discussed above.

As discussed above, the GA may be comprised of variable parameters used in mathematical relationships, which may be used to achieve a desired target function (e.g., a maximum value or a minimum value, depending on how the mathematical relationships are expressed). The GA may take into consideration a plurality of sequence-specific features derived from the nucleic-acid sequence of the target genome and/or positional specifications of primers to be used to detect the target genome's nucleic-acid sequence. The sequence-specific features may be comprised of any one or any combination of: primer-sequence composition, relative positions of primers, primer-sequence complexity, thermodynamic properties that may be relevant to reaction kinetics, etc. How the sequence-specific features are used in the GA is based on an initial primer-design algorithm, which utilizes initial values for the variable parameters and which applies those initial values to initial mathematical relationships, obtaining experimental data on the detection efficacy from primer sets designed using the initial primer-design algorithm, and refining the variable parameters and the mathematical relationships based on the experimental data. By performing multiple “design-experiment-refine” cycles, the GA may be trained to be increasingly effective in designing improved primer sets for detecting the target genome. Moreover, because different collections of optimized primers may result from multiple runs of the GA for the same parent primer set and/or from different parent primer sets, enhanced and scalable sampling of optimized primer sets may be possible. In some embodiments, the training may use PLS regression to improve target-function parameterization. In some embodiments, the parameterization may be iteratively improved through the virtuous cycle 300.

FIG. 3B schematically illustrates aspects of the virtuous cycle 300, according to some embodiments of the present technology. In some embodiments, the virtuous cycle 300 may be improved through experimental evaluation 308 and machine-learning training 310 using experimental data from the experimental evaluation 308. In some embodiments, a first-round diverse group of primer sets 302 may be generated using a design system 304 (e.g., the design system discussed above and/or the design method illustrated in FIG. 1). In some embodiments, the first-round diverse group of primer sets 302 may be used in initial clinical tests for the experimental evaluation 308 of the first-round diverse group of primer sets 302. In some embodiments, the experimental evaluation 308 may provide experimental data to a machine-learning training algorithm 310. In some embodiments, the machine learning algorithm 310 may use the experimental data to modify the design system 304 (e.g., to refine the variable parameters and/or the mathematical relationships of the GA, discussed above) to produce an improved design system 304a. The improved design system 304a may design a next-round diverse group of primer sets 302a, which may be used in clinical tests and undergo the experimental evaluation 308. Experimental data from the experimental evaluation 308 may be provided to the machine-learning training algorithm 310 for a next round of improvements to the improved design system 304a, and so on. The virtuous cycle 300 may be repeated until a primer set having a desired efficacy is obtained or until a fixed number of cycles has been completed. Optionally, other primer sets 306 designed by other platforms may be used in the initial clinical tests and/or in later clinical tests to obtain comparison data to be compared with experimental results obtained from the primer sets 302, 302a designed by the design system 304, 304a.

The techniques described herein may be used to curtail the spread of a new disease, by enabling faster and more comprehensive optimization of primer sets designed to detect the new disease. As noted above, the present technology may take into consideration potentially advantageous second-order effects that would not be possible using conventional techniques to design primer sets. Moreover, through use of machine learning to iteratively refine the variable parameters and/or the mathematical relationships of the GA, and through use of experimental data obtained from clinical testing of previous-generation primer sets designed using a previous generation of the GA, each new generation of the GA may be used to design better primer sets that may be more sensitive to the new disease and/or may detect the new disease more quickly than primer sets designed using the previous generation of the GA. For example, compared with conventional techniques, the techniques described above may be used to efficiently design a primer set that is optimized for a rapid isothermal amplification test for detecting the new disease. As noted above, such a rapid test may be administered (or self-administered) by a lay person in a non-laboratory setting, without the need to use expensive laboratory equipment and without the need to involve trained laboratory technicians. Therefore, such a rapid test may enable earlier testing of a larger number of individuals than tests requiring involvement of trained professionals and laboratory equipment. Moreover, the primer set designed according to the techniques disclosed herein may be of higher quality than conventionally designed primer sets. As will be appreciated, by enabling earlier testing of a larger number of individuals, the rapid test may lead to affected individuals seeking treatment sooner to counter the effects of the new disease and/or may lead to affected individuals being separated from the general population sooner and therefore deterring spreading of the new disease into the general population.

### 5.5 Pre-Screening Pipeline to Identify Genomic Targets

Aspects of this disclosure provide methods for determining primer sets for amplifying a target nucleic acid for a target pathogen. In some embodiments, the target pathogen may have a relatively small genome (e.g., viral pathogens) and a genome-wide approach may be used to determine primer sets. In other embodiments, the target pathogen may have a relatively large genome (e.g., bacteria pathogens) and two different approaches may be employed: (1) use of a candidate gene, and (2) use of a pre-screening pipeline. The candidate-gene approach may be used when there is knowledge of the target pathogen to identify regions of the target genome that may be used to determine primer sets (e.g., candidate genes). Alternatively, the pre-screening pipeline approach may be used if there is little or no known information to identify regions of the target genome that may be used to design primer sets, i.e., when there is not enough information to identify candidate genes. As described below, the pre-screening pipeline approach may be used prior to using the primer-set design technology disclosed herein, to identify candidate regions of the genome of the pathogen of interest, i.e., candidate genes, to be used in the primer-set design technology disclosed herein. An example of the pre-screening pipeline to identify genomic targets may be found in section 6.4 Experiment 4—Bacterial Pre-Screening Pipeline used for chlamydia and gonorrhea

In some embodiments of the present technology, the consensus sequence may be adapted from a target genome sequence using a pre-screening pipeline. The pre-screening pipeline may be applied, in a non-limiting example, to bacterial genomic targets (e.g., Chlamydia trachomatis (CT), or Neisseria gonorrhoeae (NG)). The pre-screening pipeline may identify the most favorable bacterial target gene segments based on certain characteristics (e.g., conservation among target variants, uniqueness/divergence from non-target organisms, length, G+C content).

Some aspects of the pre-screening pipeline may involve using known libraries or other information sources (collectively referred to as “libraries” herein), such as NCBI. As described herein, the term “NCBI” may refer to The National Center for Biotechnology Information, which is part of the United States National Library of Medicine, a branch of the National Institutes of Health. Aspects of the pre-screening pipeline may involve collecting assemblies from libraries. As herein described, an “assembly” may refer to a genome assembly (e.g., a single genome) . . . . In some embodiments, the assembly may be a RefSeq assembly. As described herein, the term “RefSeq” may refer to The Reference Sequence database, which is an open access, annotated and curated collection of publicly available nucleotide sequences and their protein products. In some embodiments, the assembly may be a GenBank assembly. As described herein, the term “GenBank” may refer to The GenBank sequence database, which is an open access, annotated collection of all publicly available nucleotide sequences and their protein translations. Presently, RefSeq and GenBank are part of NCBI. In some embodiments, the assembly may be a Pathogenwatch assembly. As described herein, the term “Pathogenwatch” may refer to a web-based platform for epidemiological surveillance using genome sequencing data.

In some embodiments of the present technology, the pre-screening pipeline may include but is not limited to actions comprising: (1) Collecting assemblies; (2) performing a pan genome analysis; (3) performing a plasmid identification; (4) standardizing and automating collection of target genomes; and (5) identifying homologs from closely related organisms.

### (1) Collecting Assemblies.

Genomic sequence data for the target pathogen may be available from differently sources (e.g., assemblies associated with NCBI; assemblies associated with libraries of various countries; assemblies associated with government laboratories, academic institutions, and the like; assemblies associated with global public health or global epidemiological surveillance organizations such as GISAID (Global Initiative on Sharing Avian Influenza Data); and independent assemblies associated with private institutions). In some embodiments of the present technology, pre-screening pipeline assemblies (e.g., RefSeq assemblies) may be systematically and automatically collected from libraries and/or other sources of assemblies. In some embodiments, a pre-screening pipeline may collect all RefSeq assemblies and annotations available to-date from NCBI, regardless of their assembly level (i.e. contig, scaffold, chromosome, complete genome). In some embodiments, collecting RefSeq assemblies from NCBI may involve a user input NCBI Taxonomy ID for the species of interest. In other embodiments, this step may be modified to include all GenBank assemblies (instead of or in addition to RefSeq assemblies). In other embodiments, this step may be modified to include all Pathogenwatch assemblies (instead of or in addition to RefSeq and/or GenBank assemblies). In some embodiments, this step may be bypassed. In some embodiments bypassing may be performed by creating an appropriate folder structure and placing annotated genomes within the folder structure in an appropriate format.

In one non-limiting example, genomic sequence data may be collected for CT (Chlamydia trachomatis) by automatically collecting all RefSeq assemblies and annotations available to-date from NCBI. In a variation of this example, the genomic sequence data for CT may also be collected by automatically collecting all GenBank assemblies and annotations available to-date from NCBI.

In another non-limiting example, genomic sequence data may be collected for NG (Neisseria gonorrhoeae) by automatically collecting all RefSeq assemblies and annotations available to-date from NCBI. In a variation of this example, a pre-screening pipeline for NG may be bypassed altogether by creating an appropriate folder structure and placing annotated genomes within the folder structure in an appropriate format. For example, NG genomes hosted at Pathogenwatch may be collected based on certain selection criteria (e.g., date and location the genome sequence was sampled from; sampling strategies used to collect genomes; etc.).

In a further non-limiting example, genomic sequence data may be collected for GAS (Group A Strep, also known as Streptococcus pyogenes) by automatically collecting all RefSeq assemblies and annotations available to-date from NCBI.

(2) Performing pan genome analysis

In some embodiments of the present technology, after collecting genome sequence data for a target pathogen, a pan genome analysis may be performed, to analyze all genome sequences of the collected genome sequence data. In some embodiments, a known pangenome analysis pipeline may be used to perform the pan genome analysis. In some embodiments, the pangenome analysis pipeline is a BPGA (Bacterial Pan Genome Analysis) pipeline. In some embodiments, the pangenome analysis pipeline is a Roary procedure. In some embodiments, options in the Roary procedure may be enabled such that alignment for each gene may be performed. In some embodiments, the individual gene alignments may be used as inputs for calculating diversity. In some embodiments, for every gene in the pan genome present in more than one isolate or copy, a number of segregating sites (Watterson's Theta), a pairwise nucleotide diversity (Pi), and a neutrality test statistic (Tajima's D) may be calculated. In some embodiments, Watterson's Theta, Pi, and Tajima's D may be calculated using an Egglib python package on the gene-wise alignments output in the pan genome analysis.

As described herein, the term “Watterson's Theta” may be defined as a measure of genetic diversity and represents the expected number of segregating sites observed between a pair of homologous sequences sampled from a given population.

As described herein, the term “Pi” may be defined as a measure of genetic diversity and is the average pairwise difference between all possible pairs of individuals in your sample.

As described herein, the term “Tajima's D” may be defined as a measure of genetic diversity that is computed as the difference between two measures of genetic diversity: the mean number of pairwise differences and the number of segregating sites, each scaled so that they are expected to be the same in a neutrally evolving population of constant size.

(3) Plasmid identification

In some aspects of the present disclosure, it may be desirable to design primers that target specific parts of the target genome. As described herein, the term “genome” may refer to all genetic information of an organism and consists of nucleotide sequences of DNA. The nuclear genome may include genes that do or do not code for proteins.

The genome may include chromosomal and non-chromosomal DNA. An example of non-chromosomal DNA may be a plasmid. Many bacterial genomes may contain plasmids. As described herein, the term “plasmid” may refer to an extrachromosomal DNA molecule within a cell that is physically separated from chromosomal DNA and can replicate independently. For example, the CT genome may contain a plasmid that is about 7.5 kilobases (kb). In some embodiments, it may be desirable to design primer sets that target a non-chromosomal DNA region of the genome.

A plasmid identification step may be performed to correctly annotate assemblies, collected in step (1) of the pre-screening pipeline, containing plasmids. In some embodiments, assemblies may comprise a plasmid. In some embodiments, assemblies may not comprise a plasmid. In some embodiments, the assemblies that comprise a plasmid may be separately queried. For example, a query to identify core genes amongst assemblies may require 100% of assemblies to carry the gene. The identified core genes may then be linked back to the RefSeq and Genbank annotations. In some embodiments, genes may be considered core genes or accessory genes. As described herein, a “core” gene may describe genes found to have homologs in at least 90% (e.g. at least 90%, at least 91%, at least 92%, at least 93%, at least 94%, at least 95%, at least 96%, at least 97%, at least 98%, at least 99%, at least 99.5%, at least 99.9%) of the reference genomes. In some embodiments, a “core gene” may describe genes found to have homologs in at least 90% of the reference gnomes. In some embodiments, a “core gene” may describe genes found to have homologs in at least 95% of the reference gnomes. In some embodiments, a “core gene” may describe genes found to have homologs in at least 99% of the reference gnomes. In some embodiments, a “core gene” may describe genes found to have homologs in about 90%, (e.g., 90%, 91%, 92%, 93%, 94%, 95%, 96%, 97%, 98%, 99%, 99.5%, 99.9%, 100%) of the reference gnomes. In some embodiments, a “core gene” may describe genes found to have homologs in about 90%, 91%, 92%, 93%, 94%, 95%, 96%, 97%, 98%, 99%, 99.5%, 99.9%, 100% of the reference gnomes. In some embodiments, a “core gene” may describe genes found to have homologs in about 90% of the reference gnomes. In some embodiments, a “core gene” may describe genes found to have homologs in about 95% of the reference gnomes. In some embodiments, a “core gene” may describe genes found to have homologs in about 99% of the reference gnomes. As described herein, an “accessory” gene may describe genes found to have homologs in less than 90% (e.g. less than 90%, less than 91%, less than 92%, less than 93%, less than 94%, less than 95%, less than 96%, less than 97%, less than 98%, less than 99%, less than 99.5%, less than 99.9%, less than 100%) of the reference genomes. As described herein, an “accessory” gene may describe genes found to have homologs in less than 90%, less than 91%, less than 92%, less than 93%, less than 94%, less than 95%, less than 96%, less than 97%, less than 98%, less than 99%, less than 99.5%, less than 99.9%, less than 100% of the reference genomes.

In further embodiments, gene-wise alignments of either the plasmid-encoded genes or the entire plasmid sequence may be performed independently from step (2) of the pre-screening pipeline. In some embodiments, alignment of the plasmid may be performed with MAFFT or a whole-genome aligner. In some embodiments, the whole-genome aligner is Mugsy.

(4) Standardizing and automating collection of target genomes

In some embodiments of the present technology, to generate non-variant genomes the pre-screening pipeline may need to account for errors in bacterial genome classification (i.e. spelling errors, taxonomy changes, species reclassification, etc.). To standardize and automate the collection of target genomes, a step to select representative genomes for each target genome may be performed. In some embodiments, this step may involve uploading a text file containing organisms of interest to the Taxonomy Status Report Page of NCBI and downloading results from NCBI. A program or software routine may be used to automatically cross reference an output file from the NCBI taxonomy page with a RefSeq summary file. In some embodiments, this program may select, as representative genomes, sequences annotated as ‘reference’ or ‘representative’, and, in cases where neither annotation exists, may select assemblies based on their ‘assembly level’ (see examples of assembly levels above). The program may output a file containing summary information of the selected representative genomes. In some embodiments, the output may include, for one or more of the assemblies corresponding to the selected representative genomes, an ftp site where the assembly is hosted on NCBI or RefSeq. Optionally, the program may output a second file containing a list of problematic organisms (e.g., those where a genome was not identified) that should be manually inspected.

(5) Identifying Homologs from Closely Related Organisms

A consideration when designing primer sets is that the primer sets should target the target genome of interest, but should not target other closely related genomes, for example from closely related organisms. To distinguish homologs of the target genome from closely related organisms, a list may be generated of potential cross-reactive related genomes, and related organisms within the same genus of each of the target genome species may be extracted (e.g., “Chlamydia” for CT, “Neisseria” for NG, etc.). In some embodiments, genomes of these extracted potential cross-reactive related genomes may be processed with the pan-genome reference generated from the respective within-species analysis. In some embodiments, processing may include determining the presence or absence of each gene of the target genome in the potential cross-reactive related genomes. In some embodiments, the presence or absence analysis may be used as a criterion for target selection. In some embodiments, processing may be done by analysis software. In some embodiments, the analysis software is Roary or PEPPAN. In some embodiments, the analysis software is Roary. In some embodiments, the analysis software is PEPPAN.

### 5.6 Optional Features: Optimization Using a Disruption Score

Aspects of this disclosure provide methods for determining primer sets for amplifying a target nucleic acid for a target pathogen. In some embodiments, it may be desirable to design primers sets that target genomic regions based on primer location relative to a specific feature. For example, the specific feature may be a location of an exon-exon junction, or a location relative to an intron, or locations of genomic modifications. In some embodiments, a specific feature may be a (n) insertion, deletion, or rearrangements, relative to a wild-type (i.e., unmodified) reference genome. As such, as disruption score may be used to reward a primer set that is distributed on either side of one or more coordinate point(s) corresponding to the specific feature and/or when a primer itself spans such a point.

An “exon” may be understood to be a segment of DNA that is maintained in messenger RNA (“mRNA”). An “intron” may be understood to be a non-coding segment of DNA that is spliced out during transcription and not retained in mature mRNA. DNA may be comprised of at least two components, exons and introns. mRNA may encode at least one protein (i.e., a naturally-occurring, non-naturally-occurring, or modified polymer of amino acids) and may be translated to produce the encoded protein in vitro, in vivo, in situ, or ex vivo.

According to some embodiments of the present technology, to reward a primer set that spans or is located near a coordinate point, the disruption score may have a positive value that is added to a predicted performance score of the primer set (e.g., an overall fitness score calculated for the primer set). As described in the paragraph above, the disruption score may comprise a functionality that preferentially favors a primer set based on a specific feature. In some embodiments, the disruption score may have a maximum value. For example, the maximum value of the disruption score may be in a range of 1 to 4, e.g., 2.5. In some embodiments, the disruption score is in a range of 1-4 (e.g., 1-4, 1-3, 1-2, 2-4, 2-3, 3-4). In some embodiments, the disruption score is about 1, 2, 2.5, 3, 4. In some embodiments, the disruption score is about 1. In some embodiments, the disruption score is about 2. In some embodiments, the disruption score is about 2.5. In some embodiments, the disruption score is about 3. In some embodiments, the disruption score is about 4. Optionally, a disruption score may be used to penalize a primer set based on a position of one or more primers of the primer set relative to a specific feature; in such cases, the disruption score may have a negative value that is added to the predicted performance score of the primer set.

The technology disclosed herein may be used to design LAMP primer sets that span concatenated exon-exon junctions. In some embodiments, the technology disclosed herein may be used to design primer sets to recognize mRNA and not DNA. An example of using the disruption score to design LAMP primer sets may be found below in 6.3 Experiment 3—Design of RNA-Specific Human Control LAMP Primer Sets.

In some embodiments of the present technology, the design system described herein may use a disruption score to preferentially produce one or more primer sets where at least one primer site spans an exon-exon junction. As used herein, a “primer site” may be a position on the target genome where a primer set may be located. In some embodiments, the disruption score may increase in relative value under the following conditions, from smallest to largest change in value, respectively, (a) when a primer site splits an amplicon into separate pairs, (b) when a primer site spans a junction, (c) when a primer site spans a junction near a critical end of the primer, and (d) when the primer site spans a junction near the critical end of the primer and the primer is a FIP-type or a BIP-type primer. A disruption score S(P) for a primer set P, may be calculated according to Equation 61:

$$S ( P ) = ϕ ( P ) + β ( P ) \qquad Eq . 61$$

In Equation 61, Φ(P) may represent a pseudo-count of a number of segments a concatenated sequence of primer sites is split into, and β(P) may represent a penalty term, as discussed below. In some embodiments, Φ may be calculated according to Equation 62:

$$ϕ = c ( F ) = exp \{ - {{\sum }_{i = 1}}^{N} {f}_{i} ln ( {f}_{i} ) \} \qquad Eq . 62$$

In Equation 62, fᵢ may be a fractional segment length, i.e., the length of the segment divided by the full length of the primer. In a non-limiting example, Φ=c (F)=4 if the LAMP primer set is split into 4 equal segments. In some embodiments, for each primer that spans an exon-exon junction and therefore is split into two segments, the two segments may comprise critical (f_{k∝}) and non-critical ends (f′ₖ) and may be used to compute the right-hand-side of the disruption score, i.e., the penalty term β. In some embodiments, may be computed as the sum of a primer-split pseudo-count and a distance to the non-critical end. In some embodiments, β may be calculated according to Equation 63:

$$β = {{\sum }^{8}}_{k = 1} {w}_{k} [ c ( ( {f}_{k} , {f}_{k}^{′} ) ) + {f}_{k}^{′} ] \qquad Eq . 63$$

In Equation 63, wₖ may represent weights. Non limiting examples of weights include wₖ=0.03 for F3/B3, or 0.15 for LB/LF, or 0.3 for any sub-primer in BIP/FIP.

### 6. Example Implementations

Aspects of the design system disclosed herein and described above were experimentally evaluated. In one example implementation, the design system generated primer sets for a LAMP reaction that amplified a nucleic acid for a genomic viral target, SARS-CoV-2. Primer sets designed by the design system for SARS-CoV-2 may be referred to herein as InitGen. The InitGen primer sets generated using the design system were compared to other primer sets generated by two commercially available software programs: PrimerExplorer and NEB primer design. All the primer sets were evaluated using a high-throughput screening system, RoboLAMP, which is configured to quantify sensitivity, speed, and specificity. The InitGen primer sets designed using the design system disclosed herein were found to have superior across-the-board performance compared to the other primer sets designed by PrimerExplorer and NEB primer design. Moreover, the design system was found to provide flexibility and options to improve the design of primer sets by providing the ability to generate different primer sets that may have superior performance through higher-order interactions of primers of the primer sets.

To determine the performance of optimized primer sets generated by the design system relative to other primer sets generated using commercially available software programs, an initial batch of diverse primer sets (InitGen primers) were designed for SARS-CoV-2. The InitGen primer sets provided a diverse set of data for training a model for predicting the analytical performance of a primer set. A time to positivity, Tₚ, was extracted for each of the primer sets. Tₚ may be considered to be a starting point of the amplification reactions and may be used as a parameter for evaluating the performance of primer sets. The screening system computed values for 59 sequence-specific features for 231 primer sets. The best approximate correspondence between values for the 59 features and target values for the 59 features were established using a PLS-regression technique. PLS-regression techniques are known techniques used in machine learning to derive a parsimonious linear relationship between target values and feature values.

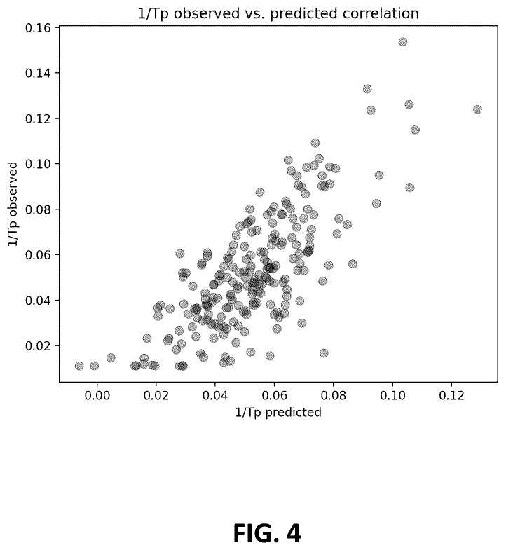

FIG. 4 shows a chart illustrating observed values vs. predicted values for inverse time-to-positivity (1/Tₚ) values for the InitGen primer sets. A correlation coefficient of R=0.6 was observed at a 10-fold cross-validation setting.

A second batch of LAMP primer sets was designed for the same SARS-CoV-2 genome target, using the design system discussed above but with updated weights and features learned from analysis of the InitGen primer sets. A resulting collection of “NextGen” primer sets was obtained after using the design system's procedures for clustering and culling the second batch of primer sets. RoboLAMP was used to evaluate the NextGen primer sets and compare them with the InitGen primer sets. The NextGen primer sets also were compared to primer sets generated using commercially available software programs.

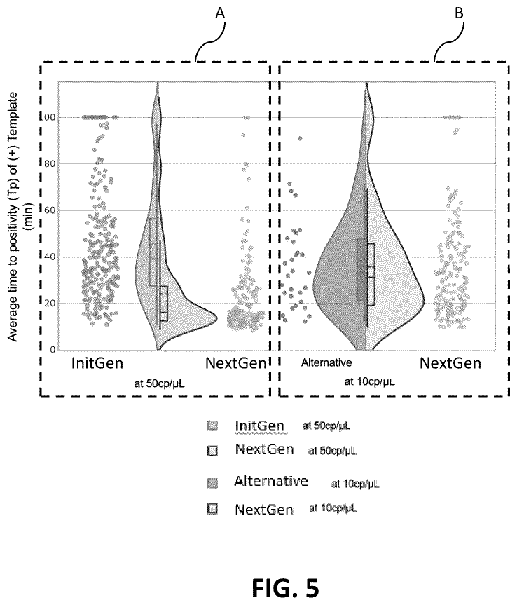

FIG. 5 shows charts illustrating an average time to positivity (Tₚ) for different collections of primer sets and samples containing different amounts of a target pathogen to be detected by the collections of primer sets, showing amplification speeds of the NextGen primer sets compared with other primer sets. Panel A of FIG. 5 shows a comparison of the Tₚ of the InitGen primer sets (data on the left) and the NextGen primer sets (darker data on the right), with all primer sets of Panel A using 50 cp/μl of target pathogen in the samples. As shown in Panel A, based on the average time to positivity (Tₚ) for the largest number of primer sets within each group of primer sets, the NextGen primer sets (darker data on the right) were found to amplify the target genome faster than the InitGen primer sets (data on the left), with the NextGen primer sets showing the largest number of positive amplifications at approximately 15 min, and with the InitGen primer sets showing the largest number of positive amplifications at approximately 30 min.

Panel B of FIG. 5 shows a comparison of the Tₚ of the NextGen primer sets (lighter data on the right) and “Alternative” primer sets (data on the left) generated using a commercially available primer-design program, with all primer sets of Panel B using 10 cp/μl of target pathogen in the samples. As shown in Panel B, based on the average time to positivity (Tₚ) for the largest number of primer sets within each group of primer sets, the NextGen primer sets (lighter data on the right) also were found to amplify the target genome faster than the Alternative primer sets (data on the left), with the NextGen primer sets showing the largest number of positive amplifications at approximately 20 min, and with the Alternative primer sets showing the largest number of positive amplifications at approximately 25 min.

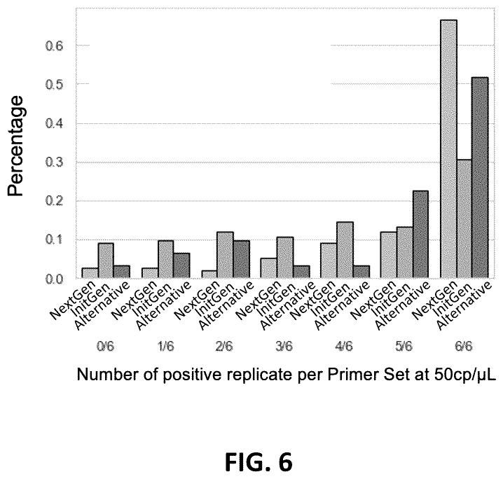

FIG. 6 shows a chart illustrating a comparison of fractions of successful amplifications for six (6) replicate experiments performed using each of the NextGen primer sets (left bar of each group of three bars), InitGen primer sets (middle bar of each group of three bars), and Alternative primer sets (right bar of each group of three bars), at 50 cp/μl of target pathogen in the samples. In FIG. 6, for each of the three types of primer sets, the chart shows the percentage of the primer set that achieved a certain fraction of successful amplifications out of the six replicates, i.e., 0/6 (0 out of 6), 1/6, 2/6, . . . or 6/6. For each of the three types of primer sets, the percentages add up to 1 for all of the fractions. As shown in FIG. 6, all of the three types of primer sets had the highest percentage of primer sets successfully amplify 6/6 (6 out of 6) replicates compared to other fractions. The NextGen primer sets were found to be more sensitive than the InitGen primer sets and the Alternative primer sets in successfully amplifying 6/6 replicates. That is, at the 6/6 fraction, the NextGen primer sets were found to amplify the target pathogen in the samples for 67% of the NextGen primer sets compared to 52% of the Alternative primer sets and 31% of the InitGen primer sets.

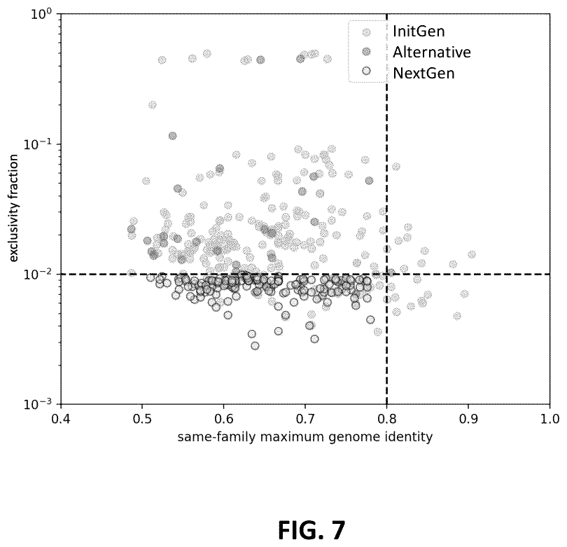

FIG. 7 shows a chart illustrating exclusivity fraction as a function of non-variant same-family maximum genome identity for InitGen primer sets (lighter circular dots), NextGen primer sets (circular dots outlined in black), and Alternative primer sets (darker circular dots). The chart of FIG. 7 shows the exclusivity fractions of the primer sets as a function of the maximum genome identity within a reference set of genomes of pathogens in the same family as the target pathogen (i.e., genomes of other coronaviruses in the same family as the SARS-CoV-2 target virus). The exclusivity fraction may be determined as one minus the inclusivity fraction calculated using Equation 25 and may represent the fraction of variants that the primer sets failed to amplify. As will be appreciated, a high inclusivity (low exclusivity) may be desirable to maximize detection of variants of the target pathogen.

In FIG. 7, a relatively lower exclusivity fraction (i.e., a higher inclusivity fraction) indicates a higher ability to detect variants of the SARS-CoV-2 target pathogen. The Alternative primer sets were not designed to be optimized for conservation, mutations, or diversity. The InitGen primer sets were designed without consideration of diversity. In contrast, the NextGen primer sets were designed to optimize for analytical performance as well as high in silico inclusivity of variants using a maximum exclusivity cut-off fraction of 0.01, which in some embodiments may correspond to excluding from detection genomes having more than one mutation in a critical position in the full primer set (see horizontal broken line at exclusivity of 10⁻² in FIG. 7). The InitGen primer sets and the Alternative primer sets were found to have relatively lower inclusivity scores (i.e., relatively higher exclusivity scores) of the primer sets studied. Comparatively, the NextGen primer sets were found to have higher inclusivity relative to the InitGen primer sets and the Alternative primer sets, and the observed exclusivity fractions of the NextGen primer sets were found to be within the 0.01 cutoff.

To minimize detection of off-target pathogens that are in the same family as the target pathogen but that are not variants of the target pathogen, a limit may be set for homology or genome identity using Equation 38. The NextGen primer sets were designed to have no more than 80% homology in any pair of primers for each sense direction in related genomes of non-variants, i.e., all known coronaviruses except SARS-CoV-1, which appears to be no longer circulating (see vertical broken line in FIG. 7). No such limit was imposed on the InitGen primer sets or the Alternative primer sets. As can be seen by the confinement of the NextGen primer sets to a quadrant within the horizontal and vertical broken lines in FIG. 7, primer sets may be designed to achieve specific inclusivity/exclusivity requirements as well as specific homology requirements, according to some embodiments of the technology presented herein.

### 6.1 Experiment 1—Construction of an Initial Dataset

An embodiment of the design system disclosed herein was used with simple settings to design an initial batch of LAMP primer sets for SARS-CoV-2. The intended use of this batch of primer sets was to train a performance prediction model (i.e., not for clinical use) so inclusivity (e.g., robustness to mutations) and cross-reactivity (e.g., with non-variants and/or background substances) were not considered. Primer sets were designed through random sampling of primers with lengths between 17 and 25 nucleotides, minimal spacings between neighboring primers, and intervals for the melting points for all primers. Eight features were computed for each primer set:
1. The average melting point of all primers as defined by PrimerExplorer;
2. The average Gibbs free energy for all primer end-caps (AG for the six terminal nucleotides in the end where the polymerase enzyme attaches);
3. rmsd-Tₘ difference average for symmetry-related primer pairs (see Equation 44);
4. Tₘ-high/low difference (see Equation 43)
5. Total amplicon length (positional distance between opposite ends of furthest primers (F3 and B3));
6. ΔG for “non-specific amplification” as described in the literature (see, e.g., Meagher R J, et al., Analyst, pp. 1924-1933, April 2018);
7. ΔG for formation of hairpins that are extendable for the complex between the two composite primers FIP and BIP, with the extendable hairpins corresponding to the two 3′-end terminal nucleotides being hybridized; and
8. Probability of formation of any hairpin between FIP and BIP.

One hundred thousand (100,000) primer-set candidates were computed along with values for all the eight features noted above. The distribution of values was derived for all features, and feature values were converted to Z-scores (values from standard normal distribution). The sign was reversed for some of the features (3, 5, and 8) to fit with our assumption that the values for all Z-scores should be maximized (re-signed and equal weights) in the search for superior performance primer sets. Finally, the total score, S, to be maximized, was calculated as the sum of all re-signed Z-scores according to Equation 59:

$$\begin{matrix}S ( Z ) = {\sum }_{i = 1}^{8} & {z}_{i} .\end{matrix} \qquad Eq . 59$$

In addition to the optimal primer set, i.e., the primer set having the highest Z-score, training of the prediction model used sub-optimal primer sets or primer sets having Z-scores below the highest Z-score. To generate a dataset that also would sample sub-optimal primer sets, some Z-scores were excluded from the sum, i.e., a sub-optimal score S′ according to Equation 60 was also considered:

$$\begin{matrix}{S}^{′} ( Z , X ) = {\sum }_{i ∈ \frac{{N}_{8}}{X}} & {z}_{i} .\end{matrix} \qquad Eq . 60$$

In Equation 60, N₈/X is the set of all integers between 1 and 8 except the numbers in the set X. Four different definitions of X={1, 2}, {3, 4}, {5, 6}, and {7, 8} were used. The one thousand (1000) primer sets with the highest full scores were identified as well as the 50 with the highest sub-optimal scores, using each of the four definitions of excluded subsets above. Primer sets having too high positional similarity were filtered out by calculating the distance between pairs of primer sets and removing the one with the lowest Z-score, producing a final batch for training containing 230 LAMP primer sets.

### 6.2 Experiment 2—Characterization of LAMP Primer Sets

Full batches of LAMP primer sets were designed for use in experimental characterization. RoboLAMP was used for the high-throughput screening (HTS) of NAAT primer sets. Oligos were ordered from IDT (Integrated DNA Technologies, Inc., Coralville, IA, US) in 96-well plates. The buffer “Master Mix” (Q5® High-Fidelity 2× Master Mix from NEB (New England BioLabs, Inc., Ipswich, MA, US)) contained (for two 384 plates reaction): 2.112 mL NEB WarmStart LAMP 2× Master Mix, 105.6 μL NEB RNAse Inhibitor (Murine), 84.48 μL NEB fluorescent LAMP dye (50×), 168.86 μL Twist Covid RNA 1,000 cp/μL (for NON-Template replace with dH₂O), 63.36 μL dH₂O (to top up). A TECAN system was used for pipetting. Template genomic material was supplied using the full genome split into 6 equally large segments. Two different concentrations of the template DNA were investigated: 50 and 1000 μM. Amplification was followed real-time with FRET detection and qLAMP for 90 minutes. Six (6) replicate experiments were performed for each sample, both with and without the target genetic material. A positive reaction was assessed through curve fitting for sigmoidal shape (see, e.g., Subramanian S et al., PLOS ONE, vol. 9, no. 6, c100596, 2014). This fitting procedure extracts the time to positivity (Tₚ), which is the time where the curve starts to rise. The melting point was determined for the reaction product of both template and non-template control (NTC) reactions to assess false positives. A candidate sample replicate was considered a false-positive result if the Tₚ of any of the NTC was below the candidate Tₚ. Experiments with complete absence of curve rise (no-shows) were assigned a large value, T_{noshow}>90 minutes, for Tₚ.

### 6.3 Experiment 3—Design of RNA-Specific Human Control LAMP Primer Sets

Full batches of LAMP primer sets of commonly used and highly expressed genes in humans were designed for use in experimental characterization. In this example, LAMP primer sets were designed for use as control primer sets that span concatenated exon-exon junctions. The LAMP primer sets that span exon-exon junctions should only amplify RNA because DNA contains introns between exons and would be much larger and disruptive to primer binding. Selection of genes was based on prior use in an approved test and on gene conservation. Four candidate genes were selected from known genes for primer design: POP7, PPIA, ACTB, and GAPDH. To design LAMP primer sets that span concatenated exon-exon junctions, the disruption score (see section 5.2b) was added to the performance score (see section 5.2) to reward the LAMP primer sets spanning exon-exon junctions. Additionally, a maximum number of exon-exon junctions were spanned and certain junction sites within a primer were favored. In two examples: (1) a junction site near middle or critical end were favored and (2) a junction site including FIP/BIP subprimers were favored.

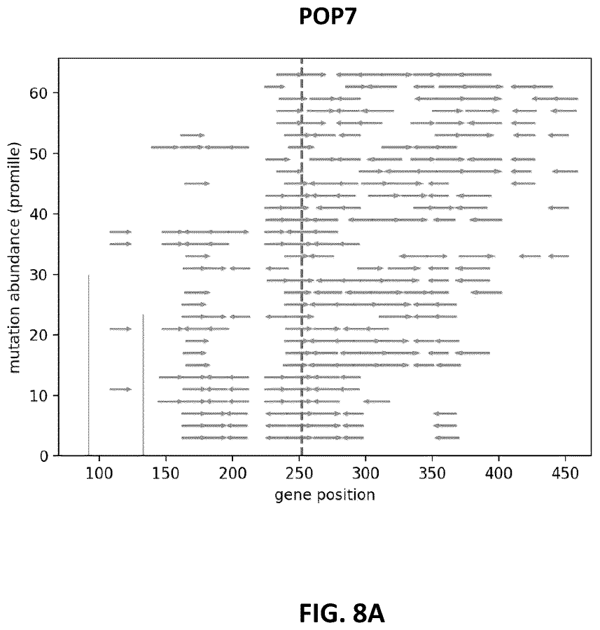

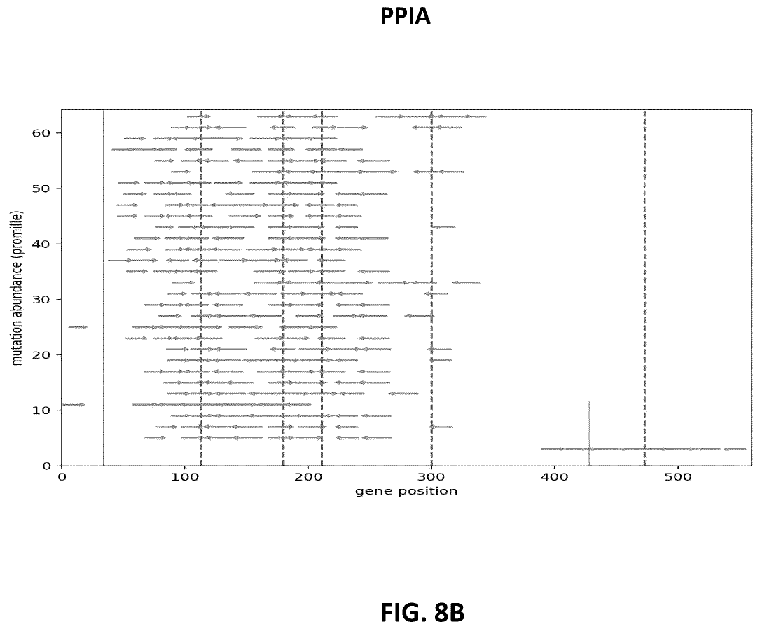

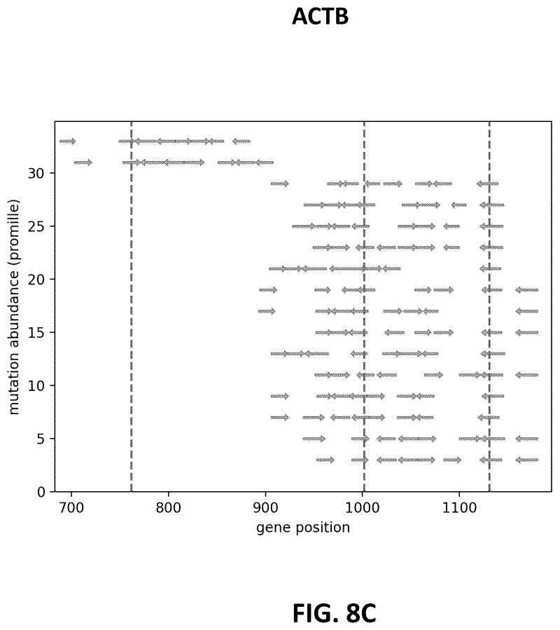

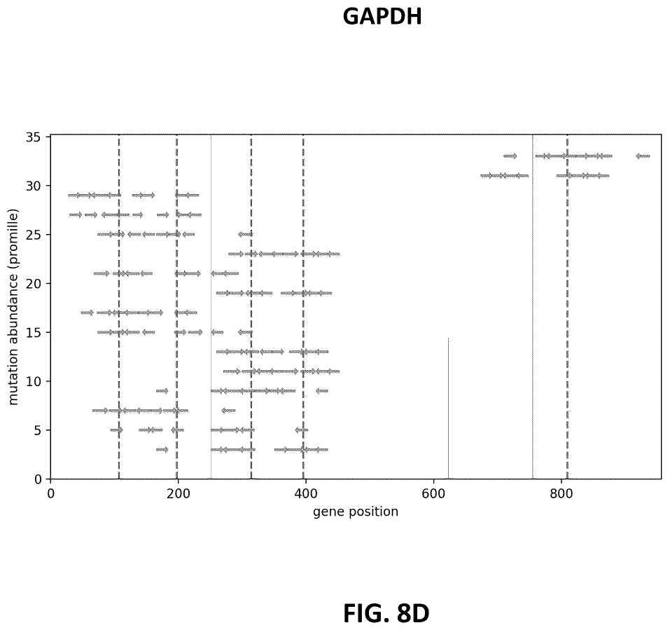

POP7 LAMP primer sets were designed from ENST00000303151.4 containing exon 1 and exon 2. Exon 1 and exon 2 are split at gene position 252. Successful design of 31 LAMP primer sets are shown in FIG. 8A. In FIGS. 8A-8D the x-axis is the position of the primer set relative to the primer set's nucleotide position on the gene without introns (position 1 represents the first coding base pair). The y-axis is the mutation abundance. Vertical dashed lines represent the gene position of exon-exon junctions. Arrows represent LAMP primers, or sub-primers for the case of FIP and BIP, and each row of arrows represents a LAMP primer set.

PPIA LAMP primer sets were designed from ENST00000355968.10. All exons were used to generate templates for primer design. Successful design of 31 LAMP primer sets are shown in FIG. 8B and span up to 4 exon-exon junctions. (See description of x-axis, vertical dashed lines, and arrow above.)

ACTB LAMP primer sets were designed from ENST00000331789.9. All exons were used to generate templates for primer design, except the untranslated region (UTR) of exon 6. The UTR of exon 6 was cropped to the CDS portion (−700 bp) due to common genetic variants including an INDEL. The gene coding ACTB is on the antisense strand. Successful design of 16 LAMP primer sets are shown in FIG. 8C. (See description of x-axis, vertical dashed lines, and arrow above.)

GAPDH LAMP primer sets were designed from ENST00000396859.5. Exons 3 to 8, which share amongst ¾ of the most highly expressed transcripts across lung, vagina, and esophagus mucosa tissues were used to generate templates for primer design. Successful design of 16 LAMP primer sets are shown in FIG. 8D and span up to 4 exon-exon junctions. (See description of x-axis, vertical dashed lines, and arrow above.)

All LAMP primer sets for all four gene candidates (POP7, PPIA, ACTB and GAPDH) have high predicted performance scores and inclusivities.

### 6.4 Experiment 4—Bacterial Pre-Screening Pipeline Used for Chlamydia and Gonorrhea

Chlamydia trachomatis (CT) and Neisseria gonorrhoeae (NG), which cause the sexually transmitted diseases chlamydia and gonorrhea, respectively, were investigated using the pre-screening pipeline from Section 5.5. The number of LAMP primer sets required to detect CT and NG with acceptable genetic inclusivity, the fraction of known variants expected to be robustly detected, was investigated. To design primer sets, the bacterial pre-screening pipeline was used to identify genomic targets in an unbiased manner, requiring no a priori knowledge of the pathogen and applied to identify candidate regions in CT and NG. LAMP primer sets for Group A Strep (GAS), aka Streptococcus pyogenes were also designed and evaluated using a candidate gene approach. The bacterial pre-screening pipeline was applied to GAS as a control.

### Methods

Collecting Assemblies from Libraries (e.g., NCBI).

A systematic and automated method of collecting all RefSeq assemblies and annotations available to-date from NCBI was created, and applied to CT (n=171), NG (n=864), and GAS (n=2185). Additionally, to increase the size of the variant database, this step was modified for CT to include all GenBank assemblies (n=342). Further additionally, this step was modified for NG to collect assemblies hosted at Pathogenwatch. From the greater than 13,000 NG assemblies available on Pathogenwatch, six collections (e.g., a group of assemblies from a specific source, study, or project) were selected based on their geographic locations and sampling strategies. The six collections were selected from NG assemblies collected within the framework of the Gonococcal Isolate Surveillance Project (GISP), the European gonococcal antimicrobial surveillance programme (Euro-GASP), and the WHO. The six collections are: Schmerer et al. (2020)-324 GISP isolates; Grad et al. (2016)-1035 GISP isolates; Thomas et al. (2019)-644 GISP isolates; Grad et al. (2014)-216 GISP isolates; WHO reference-14 reference isolates; and EuroGASP (2013)-1054 Euro-GASP isolates. The final Pathogenwatch NG dataset consisted of genomes and annotations from 2943 isolates.

### Within-Species Pan Genome Analysis and Diversity and Selection Quantification.

Following the download and reformatting of genomes and annotations, a pan genome analysis was performed for each dataset using one of most widely-used programs, Roary, with the -z -e -n -v -s -i 92 flags enabled. By enabling the -e -n and -z options, multi-fasta alignments for each gene are performed with MAFFT, and these were used as inputs for calculating diversity within the species as well as in the pre-existing LAMP Primer Design pipeline. Ninety-two percent was used for all within-species analyses reported herein, however, this parameter can be modified. For every gene in the pan genome present in more than one isolate or copy, the number of segregating sites (Watterson's Theta), the pairwise nucleotide diversity (Pi), and the neutrality test statistic (Tajima's D) were calculated using the Egglib python package on the gene-wise alignments output in the pan genome analysis.

### CT Plasmid Identification.

Bacteria often contain plasmids. An approximately 7.5 kb plasmid is a common component of the CT genome. Several NAATs have targeted regions of this plasmid. Due to its high copy number, it is indeed an attractive target. However, it appears that plasmids are not systematically or uniformly deposited in NCBI, making assessment of these targets difficult. Of the 171 RefSeq CT assemblies, only 61 were annotated with a plasmid ‘chromosome’; of the 342 CT Genbank assemblies, only 62 were annotated with a plasmid ‘region’. Thus, a separate query for ‘core’ genes among the 61 and 62 plasmid-containing isolates was done, respectively, requiring 100% of isolates to carry the locus. These genes were then linked back to the annotation.

In addition to the gene-wise alignments of plasmid-encoded genes generated by the pan genome analyses, an alignment of the entire plasmid was performed. The following search in NCBI yielded 70 complete plasmid sequences: ((“Chlamydia trachomatis” [Organism] AND plasmid [Title]) AND complete [Title]) AND RefSeq [Filter] AND Refseq [filter]. These sequences were aligned with MAFFT under two different algorithms: “mafft—maxiterate 1000—local pair” and “mafft—maxiterate 1000—globalpair”. Upon inspection, it was found that 9 out of 70 genomes did not align well. Thus, the whole genome aligner, Mugsy, was used instead. The multiple alignment format (MAF) output by Mugsy was then parsed, separating each of six blocks into individual multi-FASTA alignments for downstream primer design.

### Standardization and Automation of Cross-Reactivity Database Generation.

A list of 127 potential cross-reactive species/strains (including CT and NG) was compiled from two different FDA documents. In compiling this list, it was noted that even amongst FDA approved documentation, several spelling errors exist in organism names and that bacterial taxonomy is often subject to change (i.e., species get reclassified). An in-house script was generated to cross reference the output file from the NCBI taxonomy site with a RefSeq summary to select representative genomes quickly and reproducibly for each species/subspecies. This script selects sequences annotated as ‘reference’ or ‘representative’, and in cases where neither exists, selects assemblies based on their ‘assembly level’. The outputs were a file containing the summary information, including an FTP site where the assembly is hosted by NCBI as part of its RefSeq assemblies, for each of the selected representative genomes, and a second file listing problematic organisms (i.e., those where a genome was not identified) that should be manually inspected. For the curated list of 127 organisms, all but one had a RefSeq genome available, and a GenBank assembly for Neisseria flava was manually selected and added.

Between-Species Pan Genome Analysis to Identify Homologs from Closely Related Organisms.

The LAMP primers should target CT and NG but no other closely related species that may be found at the infection site. From the list of potential cross-reactive species/strains, those within the same genus (i.e., “Chlamydia” and “Neisseria”) of each of the target species were extracted. These genomes were processed together with the pan-genome reference generated from the respective within-species analysis, using two different pan genome analysis softwares: (1) Roary with the minimum percentage identity for blastp (-i flag) set to 80, and (2) PEPPAN with the minimum identities in BLAST search parameter (—match_identity flag) set to 0.8. For each gene of the within-species pan genome, its presence or absence in these between-species analyses was determined and used later as a filtering criteria for target selection.

### Results

### RefSeq is a Reliable Source of Assemblies.

After jointly examining all analyses, it was revealed that the non RefSeq sources produced unreliable results. From the GenBank analysis of CT, there were no genes identified amongst ≥99% of isolates, only 363 genes identified in between 95-99% of isolates, and 25,442 accessory genes. This suggests that there exist several unreliable assemblies in GenBank for CT. The 2,943 NG genomes collected from Pathogenwatch similarly gave results suggesting quality control issues of assemblies, despite their supposed uniform processing and QC. From this analysis, no genes were identified as shared amongst ≥95% of isolates, while all 1,726 genes were deemed accessory. Providing reassurance that this was not just an error of the bacterial prescreening pipeline, there were genes shared across several samples.

Chlamydia trachomatis (CT)

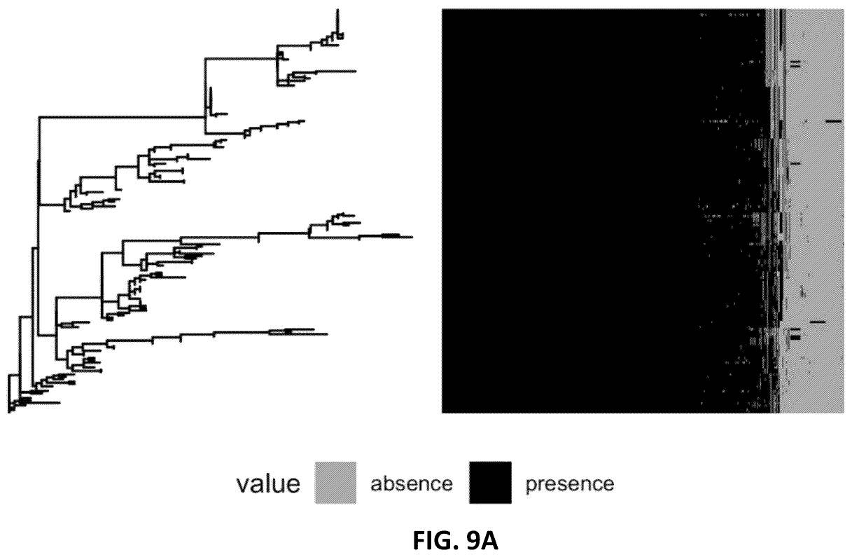

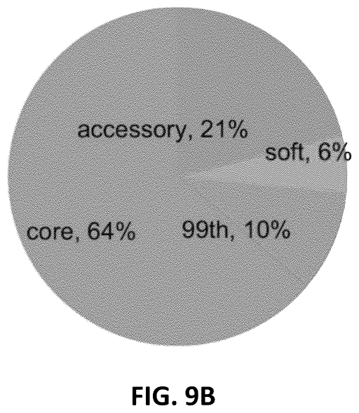

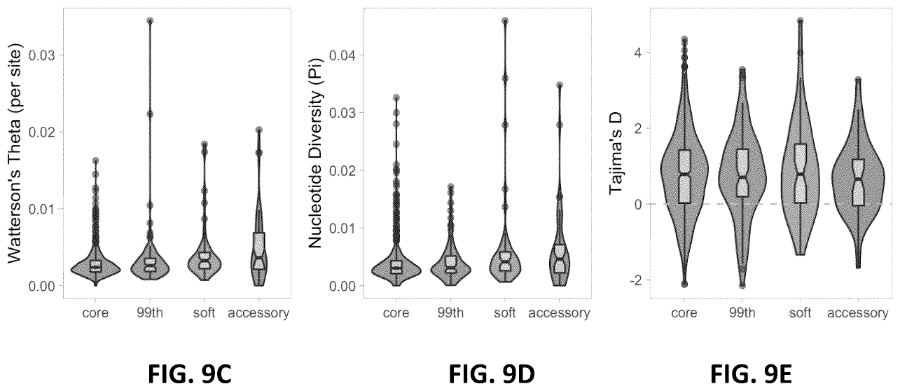

Genome-wide analysis revealed a total of 1081 genes identified amongst the 171 RefSeq CT assemblies (FIGS. 9A-9E). Of these, 982 and 684 were identified as unique to the species (i.e., they were not found to have homologs in any of the Chlamydia species included as potentially cross-reactive species) in the PEPPAN and Roary between-species pan genome analyses, respectively. These numbers drop to 750 and 457 when focusing only on genes shared by ≥99% of isolates (i.e., ‘core gene’), and further to 656 and 399 when additionally requiring 150 bp and 90% of the coding sequence to be non-missing in any strain. Because the analysis identified many core genes that are unique to the species with low diversity, this enabled fastidious selection of genomic targets for primer design.

Many commercial tests for CT target regions of the approximately 7.5 kb plasmid, and a point of care (POC) detection system using LAMP to amplify a region of the ompA gene has been previously reported. Based on this analysis, ompA did not appear to be a good target, as only 108 out of 171 isolates were found to harbor the same gene; it is possible that one of the serovars of CT is so diverse at this locus that its gene clustered as a separate locus. Additionally, this analysis highlighted another challenge in bacterial genomics: only 61/171 of the assemblies contained the plasmid sequence. Based on the literature, the plasmid is generally present in clinical isolates and thus the lack of them here most likely reflects that the submitters simply did not deposit them in NCBI at the time of the assembly submission. This is one of the many reasons an assembly pipeline was developed. To overcome the issue of not all isolates containing the plasmid sequence, focus was placed on the 61 isolates for which it was included, and the number of isolates containing each gene was identified. Additionally, an alignment of the entire plasmid based on an independent RefSeq plasmid search was created, which resulted in 70 whole plasmid sequences.

Neisseria gonorrhoeae (NG)

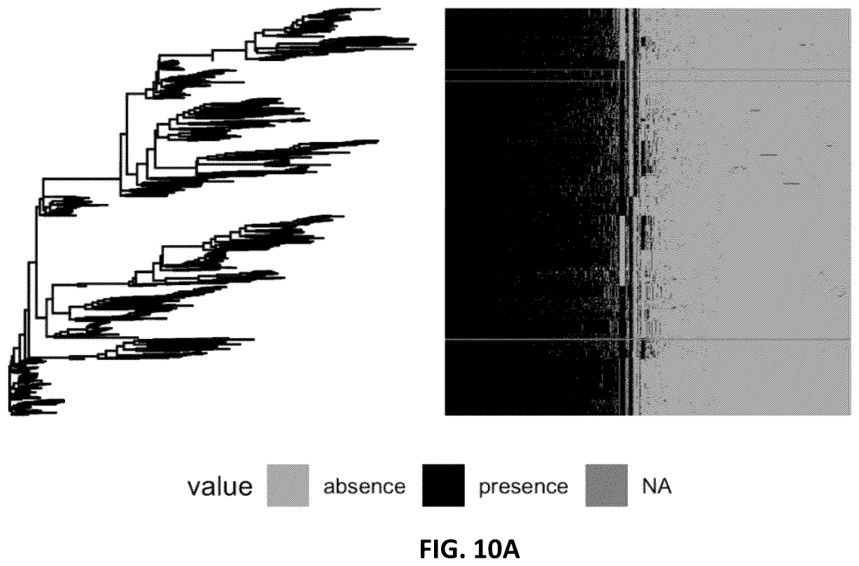

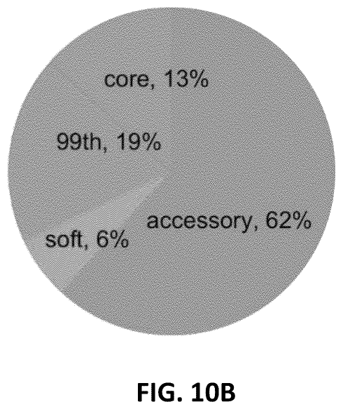

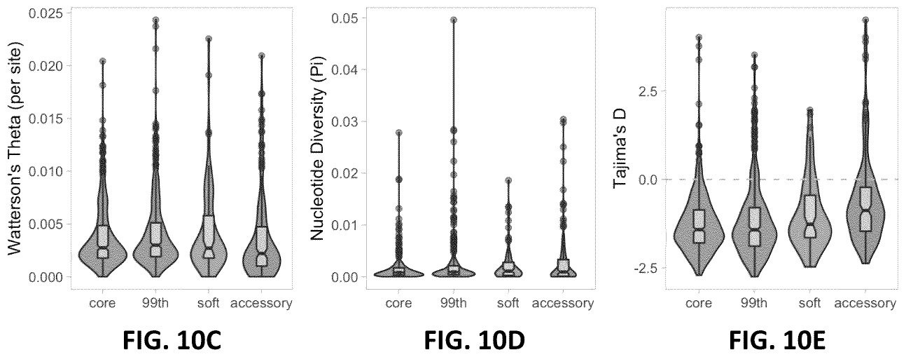

A total of 4173 genes were identified amongst 864 RefSeq NG assemblies (FIGS. 10A-10E). Of these, 567 and 1899 were identified as unique to the species in the PEPPAN and Roary analyses, respectively. These numbers drop to 42 and 41 when focusing only on genes identified in ≥99% of isolates, and further to 16 and 16 when additionally requiring 150 bp and 90% of the coding sequence to be non-missing. Of these, 14 overlap between the two analyses, with only 3 being identified in all 864 isolates. Thus, the options for selecting targets for primer design were limited in the case of NG.

Of the previously targeted regions, a highly repetitive opa target was identified in this analysis. However, it was so diverse that it aligned extremely poorly making it unusable. The penA locus was also found to have high levels of diversity and a positive (and extreme) value of Tajima's D suggesting that the locus is under balancing selection. Such high levels of diversity, however, make it an unideal target for a simple presence/absence LAMP-based test. The glnA gene looked to be a reasonable candidate region in terms of diversity, however, it had close homologs in other Neisseria species, suggesting that cross-reactivity of this region is likely to be high.

Streptococcus pyogenes, Aka Group a Streptococcus (GAS)

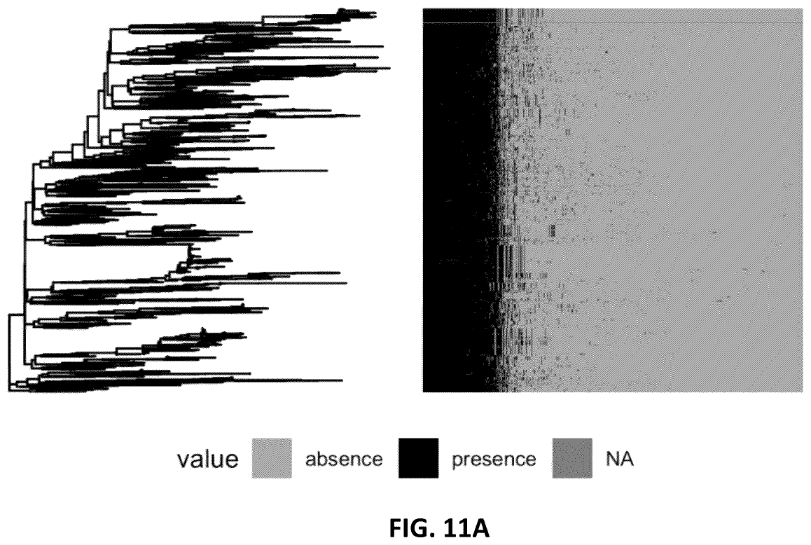

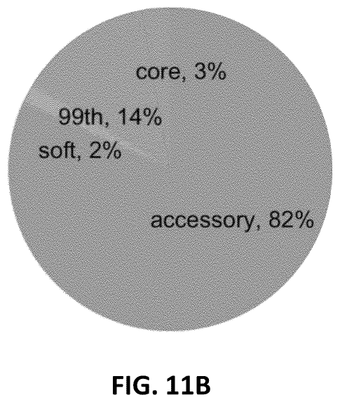

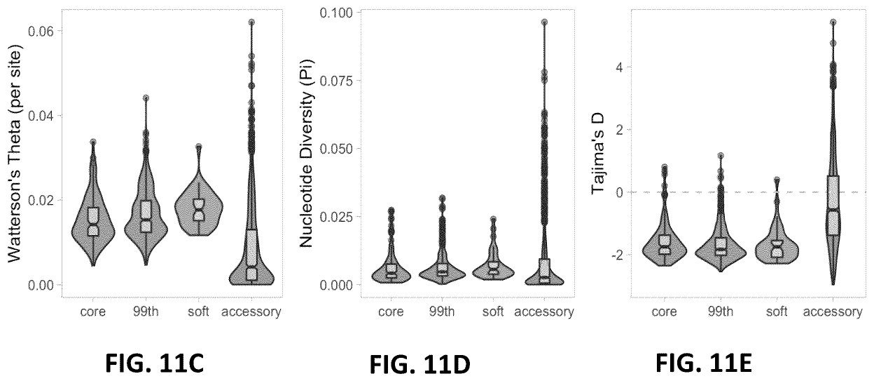

LAMP primer sets for Group A Strep (GAS), aka Streptococcus pyogenes were designed and evaluated. A candidate gene approach was used, whereby the primer design was limited to the previously targeted speB and TETR/ACrR (spy1258) loci. As validation of the newly designed bacterial pre-screening pipeline, the location where these loci landed in the genome-wide approach was investigated and additional loci that can be used to design primers were identified. A total of 7276 genes were identified among 2185 Streptococcus pyogenes genomes (FIGS. 11A-11E). Of these, 3407 and 4142 were identified as unique to the species in the PEPPAN and Roary analyses, respectively. These numbers drop to 219 and 110 when focusing only on core genes (≥99% of isolates), and further to 119 and 53 when additionally requiring 150 bp and 90% of the coding sequence to be non-missing. Of these, 52 overlap between the two analyses.

The speB locus was found in 2181 out of 2185 isolates (>99.8%) and harbored low levels of diversity. Moreover, it was not found to have homologs in any other Streptococcus species that were deemed as potentially cross reactive. All of these things point to speB as a good target for the Denali assay. In contrast, while the TETR/ACrR locus looked okay from a diversity perspective, it was found to have homologs in other Streptococcus species. Suggested primers for GAS identified using this pipeline are in Table 1.

TABLE 1
Re-design primers for GAS

| organism | gene | reason chosen | homolog |
|---|---|---|---|
| GAS | FAF78_RS07040 | 100% of isolates, no homology, low | no |
| | | diversity | |
| GAS | E9631_RS07800 | 100% of isolates, no homology, low | no |
| | | diversity | |
| GAS | E9715_RS06140 | 100% of isolates, no homology, low | no |
| | | diversity | |
| GAS | EW021_RS00280 | 100% of isolates, no homology, low | no |
| | | diversity | |
| GAS | Z472_RS05990 | 100% of isolates, no homology, low | no |
| | | diversity | |
| GAS | speB | already designed targets for . . . use new data | no |
| GAS | D3H46_RS08470 | nearly 100% of isolates, no homology, low | no |
| | | diversity | |
| GAS | FAB67_RS08845 | nearly 100% of isolates, no homology, low | no |
| | | diversity | |
| GAS | FAH02_RS03725 | nearly 100% of isolates, no homology, low | no |
| | | diversity | |
| GAS | FAP27_RS02745 | nearly 100% of isolates, no homology, low | no |
| | | diversity | |
| GAS | HRR47_RS00670 | nearly 100% of isolates, no homology, low | no |
| | | diversity | |
| GAS | Z273_RS05190 | nearly 100% of isolates, no homology, low | no |
| | | diversity | |
| GAS | FAO57_RS03340 | nearly 100% of isolates, no homology, low | no |
| | | diversity | |
| GAS | FAL74_RS03555 | nearly 100% of isolates, no homology, low | no |
| | | diversity | |
| GAS | DQM47_RS08050 | nearly 100% of isolates, no homology, low | no |
| | | diversity | |
| GAS | sdaB | 2180 samples, named gene | no |
| GAS | FAP77_RS07345 | nearly 100% of isolates, no homology, low | no |
| | | diversity | |
| GAS | D3H46_RS08470 | nearly 100% of isolates, no homology, low | no |
| | | diversity | |
| GAS | FAB14_RS04000 | nearly 100% of isolates, no homology, low | no |
| | | diversity | |

### CONCLUSIONS

A new bacterial pre-screening pipeline was developed to identify genomic target regions for at-home tests that required no a priori knowledge of the pathogen. By applying this to CT, NG, and GAS, several candidate regions were identified which can be further honed for the development of LAMP primer sets. CT and GAS yield numerous good targets, while NG was more challenging. By applying the pipeline to GAS, evidence was provided that speB is a strong candidate. Additionally, other regions that can be targeted to re-design primers for GAS was proposed.

It should be understood that the features and details described above may be used, separately or together in any combination, in any of the embodiments discussed herein.

Some aspects of the present technology may be embodied as one or more methods. Acts performed as part of a method may be ordered in any suitable way. Accordingly, embodiments may be constructed in which acts may be performed in an order different than described or illustrated, which may include performing some acts simultaneously, even though they may be shown or described as sequential acts in illustrative embodiments.

Aspects described in one embodiment may be combined in any manner with aspects described in other embodiments.

Any use of ordinal terms such as “first,” “second,” “third,” etc., in the description and the claims to modify an element does not by itself connote any priority, precedence, or order of one element over another, or the temporal order in which acts of a method are performed, but is or are used merely as labels to distinguish one element or act having a certain name from another element or act having a same name (but for use of the ordinal term) to distinguish the elements or acts.

The indefinite articles “a” and “an,” as used herein in the specification and in the claims, unless clearly indicated to the contrary, should be understood to mean “at least one.”

Any use herein, in the specification and in the claims, of the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Any use herein, in the specification and in the claims, of the phrase “equal” or “the same” in reference to two values (e.g., distances, widths, etc.) should be understood to mean that two values are the same within manufacturing tolerances. Thus, two values being equal, or the same, may mean that the two values are different from one another by +5%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. As used herein in the specification and in the claims, the term “or” should be understood to have the same meaning as “and/or” as defined above.

The terms “approximately” and “about” if used herein may be construed to mean within ±20% of a target value in some embodiments, within ±10% of a target value in some embodiments, within ±5% of a target value in some embodiments, and within ±2% of a target value in some embodiments. The terms “approximately” and “about” may equal the target value.

The term “substantially” if used herein may be construed to mean within 95% of a target value in some embodiments, within 98% of a target value in some embodiments, within 99% of a target value in some embodiments, and within 99.5% of a target value in some embodiments. In some embodiments, the term “substantially” may equal 100% of the target value.

What is claimed is:

1. A method of determining a set of primers for amplifying a target nucleic acid, the method comprising:
(a) obtaining a first primer set comprised of a plurality of primers;
(b) generating a plurality of child primer sets by performing a plurality of modifications to the first primer set;
(c) for each of the child primer sets, determining a fitness score of the child primer set; and
(d) for each of the child primer sets, if the fitness score of the child primer set is at or above a predetermined threshold, determining the child primer set to be an acceptable primer set and adding the child primer set to a first collection of acceptable primer sets stored in a memory device.

2. The method of claim 1, wherein the generating generates at least some of the child primer sets in parallel.

3. The method of claim 1, wherein the acceptable primer sets of the first collection are stored in the memory device together with corresponding fitness scores of the acceptable primer sets.

4. The method of claim 1, further comprising:
(e) outputting the first collection for use in amplifying the target nucleic acid or for use in optimization of one or more acceptable primer sets of the first collection.

5. The method of claim 1, wherein at least one of the child primer sets is generated by changing a nucleotide position of a starting point or an ending point of one or more primers of the first primer set.

6. The method of claim 1, wherein at least one of the child primer sets is generated by causing a mutation in one or more primers of the first primer set.

7. The method of claim 1, wherein at least one of the child primer sets is generated by replacing one or more primers of the first primer set with one or more primers of a collection of candidate primers.

8. The method of claim 1, wherein at least one of the child primer sets is generated by combining one or more primers of another child primer set with one or more primers of the first primer set.

9. The method of claim 1, further comprising:
clustering the acceptable primer sets of the first collection into two or more groups of acceptable primer sets, each group of acceptable primer sets being comprised of primer sets having a common characteristic that is different from a characteristic of another group of acceptable primer sets; and
for each group of acceptable primer sets, culling the primer sets of the group so that no more than four primer sets remain in the group.

10. The method of claim 1, wherein the obtaining of the first primer set is comprised of:
modifying an acceptable primer set of the first collection, or
modifying a child primer set having a fitness score below the predetermined threshold.

11. The method of claim 1, wherein the obtaining of the first primer set is comprised of selecting primers from a collection of candidate primers based on a target function of a genetic algorithm.

12. The method of claim 11, wherein the selecting of the primers is comprised of:
selecting a first primer randomly, and
for each other primer other than the first primer, selecting the other primer based on an optimization of the target function using the first primer and each already-selected other primer.

13. The method of claim 11, wherein a fitness score of a primer set being evaluated is determined by applying a plurality of parameters corresponding to the primer set being evaluated to a multi-variable scoring function that simultaneously takes into consideration any two or more properties derived from oligo sequences of the primer set being evaluated, the scoring function being a part of the genetic algorithm.

14. The method of claim 11, further comprising:
determining the collection of candidate primers based on:
a target genome sequence of the target nucleic acid, and
a plurality of variant genome sequences of a plurality of variant nucleic acids, each of the variant nucleic acids being a variant of the target nucleic acid.

15. The method of claim 14, wherein the determining of the collection of candidate primers is based on a plurality of non-variant genome sequences of a plurality of non-variant nucleic acids, the non-variant genome sequences being comprised of:
sequences belonging to a same family as the target nucleic acid and being a non-variant of the target nucleic acid, and
sequences belonging to families of common organisms unrelated to the target nucleic acid.

16. The method of claim 15, wherein the determining of the collection of candidate primers is comprised of:
determining, based on the variant genome sequences, a plurality of first conserved regions of the target genome sequence, and determining single primers corresponding to the first conserved regions,
determining, based on the non-variant genome sequences, a plurality of second conserved regions of the target genome sequence, and determining single primers corresponding to the second conserved regions, and
determining a collection of single primers that are single primers for the first conserved regions and that are not single primers for the second conserved regions, the collection of single primers being the collection of candidate primers.

17. The method of claim 1, further comprising:
preparing a pre-screening pipeline for the target nucleic acid by performing at least one of:
collecting assemblies of genome sequence data comprised of a plurality of genome sequences associated with the target nucleic acid,
performing pan genome analysis on at least some of the genome sequences of the genome sequence data to determine at least one measure of diversity,
identifying plasmids in the assemblies of genome sequence data,
selecting one or more of the genome sequences to be representative of the target nucleic acid, and preparing a summary file of information summarizing the one or more of the genome sequences selected to be representative of the target nucleic acid, and
identifying homologs of the one or more of the genome sequences selected to be representative of the target nucleic acid.

18. An apparatus for determining a set of primers for amplifying a target nucleic acid, the apparatus comprising:
a computer system comprised of at least one processor; and
a memory device coupled to the computer system,
wherein the computer system is programmed to:
(a) obtain a first primer set comprised of a plurality of primers,
(b) generate a plurality of child primer sets by performing a plurality of modifications to the first primer set,
(c) for each of the child primer sets, determine a fitness score of the child primer set, and
(d) for each of the child primer sets, if the fitness score of the child primer set is at or above a predetermined threshold, determine the child primer set to be an acceptable primer and add the child primer set to a first collection of acceptable primer sets stored in the memory device.

19. A non-transitory computer-readable storage medium storing code that, when executed by one or more processors of a computer system, implements a method of determining a set of primers for amplifying a target nucleic acid, wherein the method is comprised of:
(a) obtaining a first primer set comprised of a plurality of primers;
(b) generating a plurality of child primer sets by performing a plurality of modifications to the first primer set;
(c) for each of the child primer sets, determining a fitness score of the child primer set; and
(d) for each of the child primer sets, if the fitness score of the child primer set is at or above a predetermined threshold, determining the child primer set to be an acceptable primer set and adding the child primer set to a first collection of acceptable primer sets stored in a memory device.

20. The storage medium of claim 19, wherein the generating generates at least some of the child primer sets concurrently.
